(19) United States
(12) Patent Application Publication
(10) Pub. No.: US 2013/0143804 A1
(43) Pub. Date: Jun. 6, 2013

(54) Islet Amyloid Polypeptide Toxic Oligomer is a Biomarker of Heart or Kidney Failure in Type-2 Diabetes Mellitus

(71) Applicants: Florin Despa, Davis, CA (US); Sanda Despa, Davis, CA (US); Donald M. Bers, David, CA (US); Kenneth B. Margulies, Villanova, PA (US)

(72) Inventors: Florin Despa, Davis, CA (US); Sanda Despa, Davis, CA (US); Donald M. Bers, David, CA (US); Kenneth B. Margulies, Villanova, PA (US)

(73) Assignees: Trustees of the University of Pennsylvania, Philadelphia, PA (US); The Regents of the University of California, Oakland, CA (US)

(21) Appl. No.: 13/701,574

(22) PCT Filed: Jun. 7, 2011
(86) PCT No.: PCT/US11/39463

§ 371 Date: Dec. 3, 2012

Related U.S. Application Data
(60) Provisional application No. 61/352,301, filed on Jun. 7, 2010.

(51) Int. Cl. G01N 33/74

(52) U.S. Cl. CPC G01N 33/74
USPC 514/6.9; 435/7.1; 530/391.1; 530/391.3

(57) ABSTRACT

Methods for predicting a propensity for heart or kidney failure in a diabetic or pre-diabetic individual by determining the amount and/or molecular weight of islet amyloid polypeptide present in a sample from the individual are provided.

### CROSS REFERENCE TO RELATED APPLICATIONS

This application claims the benefit of priority under 35 U.S.C. §119(e) to U.S. Application No. 61/352,301, entitled “Islet Amyloid Polypeptide Toxic Oligomer is a Biomarker of Heart Failure in Type-2 Diabetes Mellitus,” filed Jun. 7, 2010 and is hereby incorporated by reference as though fully set forth herein.

### STATEMENT AS TO RIGHTS TO INVENTIONS MADE UNDER FEDERALLY SPONSORED RESEARCH AND DEVELOPMENT

This invention was made with Government Support under Grant No. HL030077, awarded by the National Institutes of Health. The Government has certain rights in the invention.

### BACKGROUND OF THE INVENTION

Diabetes mellitus, or diabetes, is a chronic disease that is characterized by impaired glucose regulation. Diabetes can be divided into two clinical syndromes, type 1 diabetes mellitus and type 2 diabetes mellitus. In type 1 diabetes, previously called juvenile-onset or insulin-dependent, insulin production is absent due to autoimmune pancreatic β-cell destruction. Although the pathogenesis of autoimmune β-cell destruction is not completely understood, it is believed to involve interactions between susceptibility genes, autoantigens, and environmental factors. Type 1 diabetes generally develops in childhood or adolescence and accounts for about 10% of all cases of diabetes.

In type 2 diabetes, previously called adult-onset or non-insulin-dependent, insulin production may or may not be inadequate, but the body is unable to utilize the insulin that is present to normalize glucose levels in the body. It is caused by a combination of poorly understood genetic and acquired risk factors, including high-fat diet, lack of exercise, and aging. Type 2 diabetes accounts for about 90% of the cases of diabetes around the world, and is estimated to affect more than 220 million people worldwide. Although it more commonly occurs in adults, type 2 diabetes is now becoming more common in children.

Chronic diabetes can lead to long-term complications affecting various organs, especially the heart, blood vessels, eyes, kidneys, and nerves. With respect to cardiovascular disease, diabetes dramatically increases the risk of various cardiovascular problems, including coronary artery disease with chest pain, heart attack, stroke, atherosclerosis, and high blood pressure. It is estimated that adults with diabetes have heart disease rates about 2 to 4 times higher than adults without diabetes, and that 50% of people with diabetes die of cardiovascular disease, primarily heart disease and stroke.

To date, however, there is no heart failure or cardiorenal diagnostic method or treatment specific to diabetics, even though diabetic cardiac or renal dysfunction has a poorer prognosis than cardiac or renal dysfunction in general.

### BRIEF SUMMARY OF THE INVENTION

The present invention provides for methods for predicting a propensity for heart or kidney failure in an individual. In some embodiments, the method comprises determining the amount of islet amyloid polypeptide (IAPP) oligomer in a sample from the individual; and predicting the propensity for heart failure in the individual based on the determined amount of IAPP oligomer, wherein an elevated amount of IAPP oligomer compared to normal levels indicates an increased propensity for heart failure.

In some embodiments, the sample is a blood sample. In some embodiments, the individual has type 2 diabetes. In some embodiments, the individual is pre-diabetic.

In some embodiments, the determining step comprises contacting a reagent that specifically binds IAPP oligomers to the sample; and detecting the amount of IAPP oligomers bound by the reagent. In some embodiments, the reagent is an antibody. In some embodiments, the reagent (e.g., the antibody) is linked to a solid support (e.g., as a “capture reagent”).

In some embodiments, the detecting step comprises contacting a detecting antibody that binds IAPP oligomers to the IAPP oligomers bound to the reagent; and quantifying the binding of the detection antibody to the bound IAPP oligomers. In some embodiments, the detection antibody is detectably labeled.

In some embodiments, the method comprises extracting blood from the individual.

In some embodiments, wherein it is determined that the individual has a propensity for heart failure, the method further comprises designing a treatment plan to reduce the propensity for heart failure in the individual. In some embodiments, the method further comprises administering at least one medication to the individual that reduces the propensity for heart failure or heart damage.

The present invention also provides for kits for predicting a propensity for heart failure in an individual who has type 2 diabetes or is pre-diabetic. In some embodiments, the kit comprises a solid support operably linked to a reagent that specifically binds IAPP oligomers.

In some embodiments, the reagent is an antibody. In some embodiments, the kit further comprises a detection antibody that binds to IAPP oligomers when the oligomers are bound to the reagent. In some embodiments, the detection antibody is detectably labeled. In some embodiments, the solid support comprises a sensor operably linked to one or more nanoparticles, wherein the one or more nanoparticles are conjugated to an antibody that specifically binds IAPP oligomers.

The present invention further provides for screening for agents that prevent or reduce the propensity for heart failure in an individual who has type 2 diabetes or is pre-diabetic. In some embodiments, the method comprises screening a plurality of agents for the ability:

to enhance excretion of IAPP oligomers from the body and/or

to block or interfere with the formation of IAPP oligomers.

In some embodiments, the method further comprises identifying at least one agent from the plurality that enhances excretion of IAPP oligomers from the body and/or blocks or interferes with the formation of IAPP oligomers; and administering the identified agent to an animal and measuring the ability of the agent to reduce the rate of heart failure. In some embodiments, the animal is an animal model for diabetes. In some embodiments, the animal has diabetes or is pre-diabetic.

In another embodiment, a method of treating or preventing heart failure in an individual who has type 2 diabetes or is pre-diabetic is described. The method comprises administering an effective amount of a compound that has the ability to 1) enhance excretion of IAPP oligomers from the body, ii) block or interfere with the formation of IAPP oligomers, or iii) block or interfere with the function of IAPP oligomers. In some embodiments, the compound is a polymer-based membrane sealant. In some embodiments, the polymer-based membrane sealant blocks or interferes with the function of IAPP oligomers by restoring membranes damaged by IAPP oligomers.

In another embodiment, a method for predicting a propensity for heart failure in an individual who is pre-diabetic is described. The method comprises determining the amount of islet amyloid polypeptide (IAPP) oligomer in a sample from the individual; and predicting the propensity for heart failure in the individual based on the molecular weight bands corresponding to the amount of IAPP oligomer, wherein an elevated amount of larger molecular weight IAPP oligomers compared to smaller molecular weight IAPP oligomers indicates an increased propensity for heart failure. In some embodiments, an elevated amount of smaller molecular weight IAPP oligomers indicates a likelihood of accumulating larger molecular weight IAPP oligomers. In some embodiments, the smaller molecular weight IAPP oligomers are about 12 or 16 kDa. In other embodiments, the larger molecular weight IAPP oligomers are about 32 or 64 kDa, or larger.

In some embodiments, a method comprises determining the amount of islet amyloid polypeptide (IAPP) oligomer in a sample from the individual; and predicting the propensity for kidney failure in the individual based on the determined amount of IAPP oligomer, wherein an elevated amount of IAPP oligomer compared to normal levels indicates an increased propensity for renal failure. In some embodiments, the sample is a blood sample. In some embodiments, the individual has type 2 diabetes. In some embodiments, the individual is pre-diabetic. In some embodiments, the determining step comprises contacting a reagent that specifically binds IAPP oligomers to the sample; and detecting the amount of IAPP oligomers bound by the reagent. In some embodiments, the reagent is an antibody. In some embodiments, the reagent (e.g., the antibody) is linked to a solid support (e.g., as a “capture reagent”). In some embodiments, the detecting step comprises contacting a detecting antibody that binds IAPP oligomers to the IAPP oligomers bound to the reagent; and quantifying the binding of the detection antibody to the bound IAPP oligomers. In some embodiments, the detection antibody is detectably labeled. In some embodiments, the method comprises extracting blood from the individual.

In some embodiments, wherein it is determined that the individual has a propensity for kidney failure, the method further comprises designing a treatment plan to reduce the propensity for kidney failure in the individual. In some embodiments, the method further comprises administering at least one medication to the individual that reduces the propensity for kidney failure or kidney damage.

The present invention also provides for kits for predicting a propensity for kidney failure in an individual who has type 2 diabetes or is pre-diabetic. In some embodiments, the kit comprises a solid support operably linked to a reagent that specifically binds IAPP oligomers. In some embodiments, the reagent is an antibody. In some embodiments, the kit further comprises a detection antibody that binds to IAPP oligomers when the oligomers are bound to the reagent. In some embodiments, the detection antibody is detectably labeled. In some embodiments, the solid support comprises a sensor operably linked to one or more nanoparticles, wherein the one or more nanoparticles are conjugated to an antibody that specifically binds IAPP oligomers.

The present invention further provides for screening for agents that prevent or reduce the propensity for kidney failure in an individual who has type 2 diabetes or is pre-diabetic. In some embodiments, the method comprises screening a plurality of agents for the ability:

to enhance excretion of IAPP oligomers from the body and/or

to block or interfere with the formation of IAPP oligomers.

In some embodiments, the method further comprises identifying at least one agent from the plurality that enhances excretion of IAPP oligomers from the body and/or blocks or interferes with the formation of IAPP oligomers; and administering the identified agent to an animal and measuring the ability of the agent to reduce the rate of kidney failure. In some embodiments, the animal is an animal model for diabetes. In some embodiments, the animal has diabetes or is pre-diabetic.

In another embodiment, a method of treating or preventing kidney failure in an individual who has type 2 diabetes or is pre-diabetic is described. The method comprises administering an effective amount of a compound that has the ability to 1) enhance excretion of IAPP oligomers from the body, ii) block or interfere with the formation of IAPP oligomers, or iii) block or interfere with the function of IAPP oligomers. In some embodiments, the compound is a polymer-based membrane sealant. In some embodiments, the polymer-based membrane sealant blocks or interferes with the function of IAPP oligomers by restoring membranes damaged by IAPP oligomers.

### DEFINITIONS

“Islet amyloid polypeptide” or “IAPP” is a 37-amino acid peptide hormone that is co-expressed and co-secreted with insulin by pancreatic β-cells. IAPP is a major component of amyloid deposits in pancreatic islets of patients with type 2 diabetes mellitus. See, e.g., Ohsawa et al., Biochem. Biophys. Res. Commun. 160:961-967 (1989). IAPP monomers are able to form “oligomers,” intermediate structures comprising more than one monomer of IAPP which in turn can lead to the formation of either “amyloid fibrils,” IAPP oligomers arranged in a β-pleated sheet structure that appear as non-branching fibrils by electron microscopy, or “toxic oligomers,” soluble oligomers that include spherical particles and curvilinear “protofibrils” and which can induce cell death. Kayed et al., Science 300:486-489 (2003); Haataja et al., Endocrine Rev. 29:303-316 (2008). As used herein, “toxic oligomers” comprise at least an octamer of IAPP. Without being bound to a particular theory, it is believed that IAPP toxic oligomers are not simply “pre”-amyloid fibrils, but are an off-amyloid fibril pathway form of IAPP oligomer. Haataja et al., Endocrine Rev. 29:303-316 (2008).

The term “heart failure” refers to the inability of the heart to provide sufficient blood flow to the body. Causes of heart failure include, for example, myocardial infarction, hypertension, cardiomyopathy, and valvular disorders.

The term “kidney failure” or “renal failure” is characterized, e.g., by proteinuria and/or slight elevation of plasma creatinine concentration (106-177 mmol/L corresponding to 1.2-2.0 mg/dL).

The term “propensity” as used herein refers to an increased susceptibility to experiencing heart or kidney failure in a population or subpopulation of individuals. A predisposition can be measured in comparison to a general or unstratified population.

The term “diabetes mellitus” or “diabetes” refers to a disease or condition that is generally characterized by metabolic defects in production and utilization of glucose which result in the failure to maintain appropriate blood sugar levels in the body. Diabetes may be classified as type 1 diabetes (generally due to the absence of insulin production due to autoimmune destruction of pancreatic β-cells) or type 2 diabetes (generally due to existing insulin levels in the body that are inadequate to normalize plasma glucose levels, and believed to primarily result from a condition known as “insulin resistance,” in which there is a decreased biological response to normal concentrations of circulating insulin). In some cases, diabetes may also be caused by any number of other conditions, including pregnancy. The present invention can be used with regard to any form of diabetes, to the extent that the diabetes is characterized by the presence of IAPP oligomers.

A “pre-diabetic individual,” when used to compare with a sample from a patient, as diagnosed by euglycemic clamp test or fasting glucose/glucose tolerance tests, refers to an adult with a fasting blood glucose level greater than 110 mg/dl but less than 126 mg/dl or a 2 hour post-load glucose (PG) reading of greater than 140 mg/dl but less than 200 mg/dl. A “diabetic individual,” when used to compare with a sample from a patient, refers to an adult with a fasting blood glucose level greater than 126 mg/dl or a 2 hour PG reading of greater than 200 mg/dl.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

As used herein, “specific binding,” when referring to antibody binding, refers to a binding reaction which is determinative of the presence of soluble IAPP oligomers, or toxic oligomers, in the presence of other IAPP species (i.e., soluble low molecular weight oligomers or amyloid fibrils). Thus, under designated immunoassay conditions, the specified antibodies bind to the soluble IAPP oligomers of the present invention but do not significantly bind to soluble low molecular weight IAPP species or amyloid fibrils. “Low molecular weight IAPP,” as used herein, refers to IAPP species that are less that about 40 kD, which corresponds to the approximate size of an IAPP octamer. Accordingly, a soluble IAPP oligomer, or toxic oligomer, of the present invention has a molecular weight of at least about 40 kD and includes oligomers that are octamers or larger, while low molecular weight IAPP species include IAPP monomers, dimers, and tetramers. Typically, a specific or selective reaction will be at least twice the background signal or noise and more typically more than 10 to 100 times background or more. In some embodiments, an antibody that specifically binds soluble IAPP oligomers binds to the soluble IAPP oligomers at least about 10-fold, about 100-fold, about 200-fold, about 500-fold, or about 1000-fold or more than it binds low molecular weight IAPP species or amyloid fibrils.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but which functions in a manner similar to a naturally occurring amino acid.

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

The term “effective amount” means an amount of a compound according to the invention which, in the context of which it is administered or used, is sufficient to achieve the desired effect or result.

The term “compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation tumor cell proliferation. The term “function of IAPP oligomers” refers to the toxicity associated with IAPP oligomers, which can include, but is not limited to, membrane destabilization.

The term “larger molecular weight IAPP oligomers” refers to IAPP oligomers that have a molecular weight of about 32 or 64 kDa, and are made up mostly of IAPP octamers and 16-mers, respectively.

The term “smaller molecular weight IAPP oligomers” refers to IAPP oligomers that have a molecular weight of about 12 or 16 kDa, and are made up mostly of IAPP trimers and tetramers, respectively.

The term “polymer-based membrane sealant” refers to a synthetic surfactant having the ability to be inserted into a cell membrane to affect the membrane surface pressure in a manner that repairs or prevents damage resulting from membrane permeabilization.

### BRIEF DESCRIPTION OF THE DRAWINGS

FIG. 1 illustrates the detection of IAPP oligomers in serum samples. (A) Toxic oligomers were detected in serum samples from type 2 diabetes mellitus (“T2DM”) and obese (BMI>32) patients by using an antibody specific for toxic oligomers (A11) on dot blots. (B) High molecular weight islet amyloid polypeptide (“IAPP”) species (˜25 kDa) are markedly abundant in T2DM and obese (BMI>32) individuals compared to control (non-diabetic, BMI<28) individuals. (C) Increase in high molecular weight IAPP species, shown by the average increase of oligomer-specific (A11) and anti-IAPP specific immunoreactivity signals in T2DM individuals (left panel) and obese individuals (right panel). The averages are the results of integration of dot blots and bands.

FIG. 2 illustrates the presence of IAPP deposition in failing diabetic hearts demonstrated by immunohistochemistry with an anti-IAPP antibody on thin heart sections. IAPP plaques (A,B) and fibrillar deposits (C) are shown in sections from a failing heart from a diabetic patient. (D) Positive control for IAPP accumulation in a pancreas from a diabetic patient. (E) Left ventricle section from a non-failing heart; no IAPP deposits are revealed.

FIG. 3 illustrates the quantification of IAPP deposition in plaques and fibrils. (A) Dot blots with an anti-IAPP antibody in post-treatment versus pre-treatment samples. (B) Quantification of post-treatment versus pre-treatment samples.

FIG. 4 illustrates the presence of toxic oligomers amyloidogenic entities within the heart in patients with overweight/obesity (OW/OB) and diabetes (DM).

FIG. 5 illustrates IgG removal efficiently decreases the cross-reactivity with secondary antibody.

FIG. 6 illustrates the assessment of the levels and characteristic size distributions of soluble IAPP oligomers accumulated in left ventricles in pathologically distinct groups.

FIG. 7 illustrates IAPP oligomer accumulation in the heart of rats expressing human IAPP. (A) HIP rats (rats transgenic for human IAPP) demonstrate accumulation of toxic oligomers in the heart, for both T2DM rats and pre-T2DM rats. (B) Increased accumulation of toxic oligomers correlates with the increase of IAPP measured in heart protein homogenates in rats. (C) Increased accumulation of toxic oligomers correlates with the increase of IAPP, measured in heart protein homogenates after treatments with formic acid and guanidine hydrochloride to break apart the preamyloid oligomers.

FIG. 8 illustrates that exogenous IAPP oligomers increase Ca²⁺ transient amplitude in isolated rat cardiac myocytes.(A) Representative experiments in a control (top panel) myocyte and a cell pre-incubated with 50 μM hIAPP (bottom panel). (B-C) Effect of rat (B) and human (C) IAPP on Ca²⁺ transient amplitude. At 5 μM, when both rat and human IAPP are in monomeric form, they induce a modest increase in Ca²⁺ transient amplitude. Increasing the concentration of the non-amyloidogenic rat IAPP to 50 μM had no further effect on Ca²+ transients. However, at 50 μM human IAPP forms rapidly oligomers, and this resulted in a marked rise in Ca²⁺ transient amplitude. For each group, measurements were done on ≧6 myocytes from 3 different rats.

FIG. 9 illustrates that IAPP oligomers accumulate in the heart of HIP rats. (A) Dot blots with the anti-IAPP antibody comparing total IAPP level in HIP vs. UCD-T2DM rats. Dots on the left show positive controls using recombinant human (hIAPP) and rat (rIAPP); 5 ng for both. The antibody binds rIAPP with about 10x higher affinity than hIAPP. (B) Representative western blot with anti-IAPP primary antibody on ventricular myocyte lysates from pre-diabetic HIP rats, and left ventricle protein homogenates from pre-diabetic (PD) and diabetic (DM) HIP rats. High molecular weight IAPP bands are evident in all groups, indicating that IAPP accumulates in the heart starting from pre-diabetes. (C) Representative western blot with the anti-IAPP primary antibody of serum samples from HIP rats.

FIG. 10 illustrates that altered Ca²⁺ cycling in cardiac myocytes from pre-diabetic HIP but not pre-diabetic UCD-T2DM rats. Representative Ca²⁺ transients in myocytes from control (Ctl) and pre-diabetic (PD) HIP rats paced at 0.5 Hz (A) and 2 Hz (B). (C) Normalized Ca²⁺ transients in myocytes from control and pre-diabetic HIP rats (0.5 Hz) indicate slower Ca²+ transient relaxation in pre-diabetic HIP rats vs. control. (D) Mean amplitude of Ca²⁺ transients recorded in cardiac myocytes from control rats (20 myocytes, 4 rats) and pre-diabetic HIP rats (18 cells, 4 rats) paced at 0.2, 0.5, 1 and 2 Hz. At 0.2 and 0.5 Hz, Ca²⁺ transient amplitude is significantly larger in myocytes from pre-diabetic HIP rats vs. control. This difference disappears at higher stimulation frequencies. *P<0.05. (E) Mean amplitude of Ca²⁺ transients in myocytes from control rats (22 myocytes, 6 rats) and pre-diabetic UCD-T2DM rats (21 cells, 4 rats) paced at 0.2, 0.5, 1 and 2 Hz.

FIG. 11 illustrates slower Ca²⁺ transient relaxation and elevated diastolic [Ca²⁺]ᵢ in myocytes from pre-diabetic HIP rats but not pre-diabetic UCD-T2DM rats. (A) Ca²⁺ transient decay time in cardiac myocytes from control (Ctl) and pre-diabetic HIP rats (PD) paced at 0.5 Hz. (B) Ca transient decay time in myocytes from control and pre-diabetic UCD-T2DM rats paced at 0.5 Hz. (C) Diastolic [Ca²⁺]ᵢ in cardiac myocytes from control rats and pre-diabetic HIP rats paced at 0.2, 0.5, 1 and 2 Hz. At higher frequencies, diastolic [Ca²⁺]ᵢ is significantly higher in myocytes from pre-diabetic HIP vs. control rats. (D) Diastolic [Ca²⁺]ᵢ in myocytes from control and pre-diabetic UCD-T2DM rats paced at 0.2, 0.5, 1 and 2 Hz. *P<0.05

FIG. 12 illustrates reduced SERCA and increased BNP level in pre-diabetic HIP rats. (A) Alterations in the protein expression of SERCA, phospholamban and Na/Ca exchanger in hearts from pre-diabetic (PD) and diabetic (DM) HIP rats vs. control, non-diabetic rats (Ctl). (B) Increased expression of the hyperthrophic marker BNP in hearts from pre-diabetic and diabetic HIP rats. Ctl—5 hearts; PD—5 hearts, DM—5 hearts. (C) SERCA expression is unchanged in hearts from pre-diabetic UCD-T2DM. (D) BNP level is elevated in diabetic but not in pre-diabetic UCD-T2DM rats. Ctl—5 hearts; PD—5 hearts, DM—5 hearts.

FIG. 13 illustrates EM images of cardiac myocytes. Control (A-B) vs. incubated with 50 μM IAPP oligomers for 36 hours (C-H).

FIG. 14 illustrates that incubation of cardiac myocytes with IAPP oligomers does not induce significant calcein leak.

FIG. 15 illustrates incubation of cardiac myocytes with exogenous IAPP oligomers (hIAPP) and Poloxamer 188 reduces the alteration of Ca cycling.

FIG. 16 illustrates distribution of sarcolemma defect depths derived from AFM data. Density of thin sarcolemma patches is higher on cardiac myocytes incubated with IAPP oligomers. Incubation of cardiac myocytes with P188 and IAPP oligomers prevents sarcolemma damage.

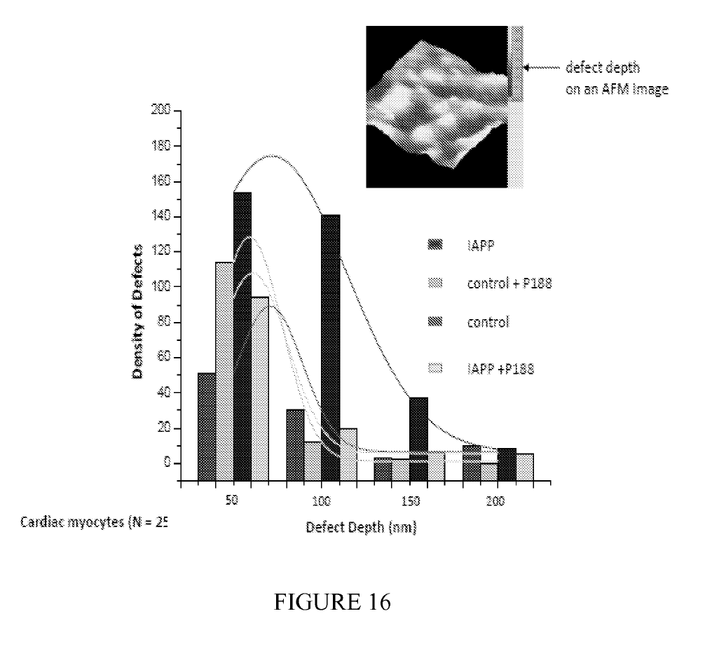

FIG. 17 illustrates the assessment of the levels of soluble IAPP oligomers in the blood of overweight/obesity (OW/OB) and diabetes (DM) patients with kidney failure.

FIG. 18 illustrates the presence of IAPP deposition in the kidney demonstrated by immunohistochemistry with an anti-IAPP antibody on thin kidney sections.

FIG. 19 illustrates the study design to determine the implications of IAPP toxic oligomer in diabetic heart failure and kidney failure.

FIG. 20 illustrates the direct and indirect implications of IAPP toxic oligomer in diabetic heart failure and kidney failure.

### DETAILED DESCRIPTION OF THE INVENTION

### Introduction

Islet amyloid polypeptide (“IAPP”; also known in the field as “amylin”), a hormone co-secreted with insulin by the pancreatic β-cells, is one of more than 20 different amyloidogenic proteins. Dobson C M. Trends Biochem. Sci. 24:329-332 (1999). These proteins are associated with life-threatening diseases, such as type-2 diabetes (Hoppener, J. W. M. et al., N. Engl. J. Med. 343: 411-419 (2000); Haataja L. et al., Endocr Rev. 29(3):303-16 (2008), neurodegenerative disorders (Selkoe, D. J., Nature 426:900-904 (2003)) and heart failure (Sanbe A. et al., Proc Natl Acad Sci USA. 101:10132-10136 (2004)). IAPP blood levels are elevated in patients at high risk of developing type-2 diabetes mellitus (T2DM), such as obese/insulin resistant individuals (Enoki S. et al., Diabetes Res Clin Pract. 15:97-102 (1992); Permert, J. et al., N. Engl. J. Med. 330:313-318 (1994); de Koning E J. et al., J Pathol. 175:253-8(1995); Young A A, Curr Opin Endocrinol Diabetes 4:282-90 (1997); Leckström A. et al. Biochem Biophys Res Commun. 239:265-8 (1997)), in patients with chronic renal failure (de Koning E J. et al., J Pathol. 175:253-8(1995); Leckström A. et al. Biochem Biophys Res Commun. 239:265-8 (1997); Ludvik B. et al, Diabetes 40:1615-9 (1991)), and in pancreatic cancer patients (Permert, J. et al., N. Engl. J. Med. 330:313-318 (1994)). At increased concentration, IAPP forms amyloids, a property that is common to this class of proteins (Dobson C M. Trends Biochem. Sci. 24:329-332 (1999); Selkoe, D. J., Nature 426:900-904 (2003); Despa F and Berry R S, Biophys. J 92 373-378 (2007); Despa F. et al. J. Biol. Phys. 34 577-590 (2008)). 96% of T2DM patients stain positive for islet amyloids derived from IAPP ((Höppener, J. W. M. et al., N. Engl. J. Med. 343: 411-419 (2000); Weyer C. et al., J Clin Invest.104:787-94 (1999); Butler A E et al., Diabetes 52:102-10 (2003)). Recently, IAPP amyloids were found within kidneys in T2DM humans, suggesting that IAPP can deposit in organs other than the pancreas. Gong W. et al., Kidney International 72:213-218 (2007).

There is accumulating clinical and experimental evidence demonstrating that toxic effects to cells in amyloidogenic diseases are actually mediated by soluble oligomeric intermediates (Haataja L. et al., Endocr Rev. 29(3):303-16 (2008); Selkoe, D. J., Nature 426:900-904 (2003); Sanbe A. et al., Proc Natl Acad Sci USA. 101:10132-10136 (2004); Haass C. et al, Nat. Rev. Mol. Cell Biol. 8:101-112 (2007); Kirkitadze, M. D. et al. J Neurosci Res. 69:567-577 (2002); Kayed R., et at. Science 300:486-489 (2003); Pattison J. S. et al. Heart Failure Circulation 117:2743-2751 (2008); Meier J. J. et al, Am J Physiol. 291:E1317-E1324 (2006)). In T2DM, IAPP toxic oligomers form intracellularly (Gurlo T. et al. Am J Pathol. 176(2):861-9 (2010)), a process that may be favored by prolonged hyperglycemic stimulations of the β-cells (Despa F., 140:115-121(2009); Despa F., Biophys. J. 98 1641-1648 (2010)). These toxic oligomer entities were shown to disrupt cellular membranes (Gurlo T. et al. Am J Pathol. 176(2):861-9 (2010)) and to alter Ca homeostasis (Huang C J et al., J Biol Chem. 285:339-48 (2010)), leading to β-cell dysfunction and apoptosis. Analysis of pancreatic islets from T2DM humans identified IAPP toxic oligomers in the intercellular space (Gurlo T. et al. Am J Pathol. 176(2):861-9 (2010)), demonstrating that intracellularly formed oligomers can be released from β-cells.

The present invention surprisingly demonstrates that islet amyloid polypeptide (“IAPP”) oligomer, a toxic amyloidogenic entity formed intracellularly in pancreatic β-cells, is present in significantly increased levels in the heart tissue of pre-diabetic and diabetic subjects. Despa S. et al, Circulation 120:S457 (2009). The present invention also surprisingly demonstrates that IAPP oligomer is present in significantly increased levels in the blood of pre-diabetic and diabetic subjects. The present invention also surprisingly demonstrates that higher molecular weight IAPP oligomer is present in pre-diabetic individuals with heart failure, resulting in a higher resolution determination of an individual's propensity for heart failure based on the molecular size of the IAPP oligomers present. Without intending to limit the scope of the invention, it is believed the mechanism by which IAPP oligomers induce heart dysfunction at the cellular level is by affecting the contractility of cardiac myocytes. The present invention further demonstrates that IAPP oligomers are present in significantly increased levels in the kidney of pre-diabetic and diabetic subjects. Accordingly, methods of predicting a propensity for heart or kidney failure in pre-diabetic and/or diabetic subjects by determining the amount of IAPP oligomer present, and methods of reducing propensity for heart or kidney failure in said subjects, are provided. The present invention further provides kits for detecting IAPP and methods for identifying agents that interfere with IAPP oligomer formation and/or enhance excretion of IAPP oligomers from the body.

### Methods for Predicting Propensity for Heart or Kidney Failure in Individuals Having Diabetic Conditions

In one aspect, the invention provides for a method for predicting a propensity for heart or kidney failure in an individual who has diabetes or is pre-diabetic, the method comprising determining the amount of islet amyloid polypeptide (IAPP) oligomer in a sample from the individual; and predicting the propensity for heart or kidney failure based on the determined amount of IAPP oligomer, wherein an elevated amount of IAPP compared to normal levels indicates an increased propensity for heart or kidney failure.

### Propensity for Heart or Kidney Failure

Diabetic patients have an increased propensity for developing heart failure as compared to non-diabetic patients, even after adjusting for age, blood pressure, weight, cholesterol, and coronary artery disease (Kannel and McGee, JAMA 241:2035-2038 (1979); Ho et al., J. Am. Cardiol. 22:6A-13A (1993)). Diabetic patients also have an increased propensity for developing kidney failure as compared to non-diabetic patients. “Heart failure,” as used herein, refers to the inability of the heart to provide sufficient blood flow to the body. Heart failure can be caused by any of a number of diseases or conditions, including but not limited to abnormal heart rhythm, myocardial infarction, coronary artery disease, hypertension, valvular disorders or abnormal heart valves, and cardiomyopathy. Symptoms of heart failure include, for example, shortness of breath, persistent coughing or wheezing, edema, fatigue, lack of appetite, nausea, confusion, impaired thinking, and increased heart rate. Kidney failure is a condition in which the kidneys lose the ability to filter toxins and waste products from the blood. Kidney failure causes abnormal fluid levels in the body, deranged acid levels, abnormal levels of potassium, calcium, and phosphate; as well as anemia, hematuria, and proteinuria.

Predicting a propensity for heart or kidney failure involves determining the amount of IAPP oligomer in a patient or patient sample and then comparing the level to a baseline or range. Typically, the baseline value is representative of levels of IAPP oligomer in a healthy person not suffering from, or likely to develop, heart or kidney failure, as measured using a biological sample such as a blood sample, other fluid sample, or tissue sample (such as heart or pancreatic tissue). Variations of levels of IAPP oligomer from the baseline range (i.e., levels of IAPP oligomer that are higher than the baseline level) indicate that the patient has an increased propensity or risk of developing heart or kidney failure or an increased risk of its recurrence.

In some embodiments, the propensity in pre-diabetic individuals is measured by evaluating the molecular weight of the IAPP oligomer. In pre-diabetic individuals an accumulation of larger molecular weight IAPP oligomers is indicative of a high propensity of heart failure. In pre-diabetic individuals an accumulation of smaller molecular weight IAPP oligomers is not indicative of a propensity of heart failure. In other embodiments, an elevated amount of smaller molecular weight IAPP oligomers in pre-diabetic individuals indicates a likelihood of accumulating larger molecular weight IAPP oligomers.

In some embodiments, the comparing step involves computer-based calculations and tools. The tools are advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

In some embodiments, the methods comprise recording a result relating to the propensity for heart failure determined from an individual. Any type of recordation is contemplated, including electronic recordation, e.g., by a computer.

### Diabetic Conditions Subject to the Methods

The methods of the present invention find use in any subject, human or non-human animal (e.g., pig, horse, birds including domestic birds, or other animals, especially those used in animal models such as mouse, rat, ferret, or non-human primate) having a diabetic condition. Diabetic conditions include, for example, type 1 diabetes mellitus, type 2 diabetes mellitus, gestational diabetes, pre-diabetes, hyperglycemia, and metabolic syndrome.

In some embodiments, the subject has type 2 diabetes. Type 2 diabetes is generally characterized by metabolic defects in production and utilization of glucose which result in the failure to maintain appropriate blood sugar levels in the body. A subject having type 2 diabetes may or may not also exhibit diabetic complications, such as damage to the nerves, blood vessels, heart, feet, kidneys, and eyes. In some embodiments, the subject is pre-diabetic. Pre-diabetes is generally characterized by impaired glucose tolerance, and frequently, although not always, precedes the onset of diabetes in a subject.

A diagnosis of diabetes or pre-diabetes can be made using any of a number of assays known in the field. Examples of assays for diagnosing or categorizing an individual as diabetic or pre-diabetic include, but are not limited to, a glycosylated hemoglobin (HbA1c) test, a connecting peptide (C-peptide) test, a fasting plasma glucose (FPG) test, an oral glucose tolerance test (OGTT), and a casual plasma glucose test. Thresholds for identifying or diagnosing an individual as pre-diabetic or diabetic using the above-described assays are readily ascertainable to one of skill in the art. For example, using the FPG test, a subject is diagnosed as having diabetes if the subject has a fasting blood glucose level greater than 126 mg/dl or a 2 hour post-load glucose reading of greater than 200 mg/dl; a subject is diagnosed as having pre-diabetes using the FPG test if the subject has a fasting blood glucose level greater than 110 mg/dl but less than 126 mg/dl or a 2 hour post-load glucose reading of greater than 140 mg/dl but less than 200 mg/dl.

### Methods of Detecting IAPP Oligomers

In some embodiments, the step of determining the amount of IAPP oligomers in a sample comprises contacting a reagent that specifically binds IAPP oligomers to the sample, and detecting the amount of IAPP oligomers bound by the reagent. In some embodiments, the detecting step comprises contacting a detection antibody that binds IAPP oligomers to the IAPP oligomers bound to the reagent; and quantifying the binding of the detection antibody to the bound IAPP oligomers.

IAPP oligomers can be detected using any of a number of well-known immunological binding assays (see, e.g., U.S. Pat. Nos. 4,366,241; 4,376,110; 4,517,288; and 4,837,168). For a review of the general immunoassays, see also Asai Methods in Cell Biology Volume 37: Antibodies in Cell Biology, Academic Press, Inc. NY (1993); Stites, supra. Immunological binding assays (or immunoassays) typically utilize a “capture agent” to specifically bind to and often immobilize the analyte (i.e. IAPP oligomers). In some embodiments, the capture agent is a moiety that specifically binds to the analyte. The antibody may be produced by any of a number of means well known to those of skill in the art and as described above. The capture agent can also be, for example, a non-antibody protein having affinity for IAPP oligomers. Examples of non-antibody affinity proteins include, but are not limited to, avimers, adnectins (see, e.g., U.S. Pat. No. 6,818,418), and anticalins (see, e.g., Beste et al., Proc. Natl. Acad. Sci. U.S.A. 96(5):1898-1903 (1999)).

Immunoassays also often utilize a labeling agent to bind specifically to and label the binding complex formed by the capture agent and the analyte. The labeling agent may itself be one of the moieties comprising the antibody/analyte complex. Alternatively, the labeling agent may be a third moiety, such as another antibody, that specifically binds to the antibody/protein complex.

In some embodiments, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111:1401-1406 (1973); and Akerstrom, et al. J. Immunol., 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

### Non-Competitive Assay Formats

Immunoassays for detecting IAPP oligomers from biological samples, such as blood and heart tissue, may be either competitive or noncompetitive. Noncompetitive immunoassays are assays in which the amount of captured protein or analyte is directly measured. In one preferred “sandwich” assay, for example, the capture agent (e.g., antibodies specific for the IAPP oligomers of the invention) can be bound directly to a solid substrate where it is immobilized. These immobilized antibodies then capture the IAPP oligomers present in the test sample. The IAPP oligomers of the invention thus immobilized are then bound by a labeling agent, such as a second labeled antibody specific for the polypeptide. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

### Competitive Assay Formats

In competitive assays, the amount of protein or analyte present in the sample is measured indirectly by measuring the amount of an added (exogenous) protein or analyte displaced (or competed away) from a specific capture agent (e.g., antibodies specific for IAPP oligomers of the invention) by the protein or analyte present in the sample. The amount of immunogen bound to the antibody is inversely proportional to the concentration of immunogen present in the sample. In a particularly preferred embodiment, the antibody is immobilized on a solid substrate. The amount of analyte may be detected by providing a labeled analyte molecule. It is understood that labels can include, e.g., radioactive labels as well as peptide or other tags that can be recognized by detection reagents such as antibodies.

### Other Assay Formats

In some embodiments, dot blot or western blot (immunoblot) analysis is used to detect and quantify the presence of IAPP oligomers of the invention in a sample. The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the IAPP oligomers. For example, antibodies are selected that specifically bind to the IAPP oligomers of the invention on the solid support. These antibodies may be directly labeled or alternatively may be subsequently detected using labeled antibodies (e.g., labeled sheep anti-mouse antibodies) that specifically bind to the antibodies against the IAPP oligomers of interest.

In some embodiments, non-antibody antigen binding molecules are used in assays to detect and/or quantify the presence of IAPP oligomers of the invention in a sample. Exemplary non-antibody antigen binding molecules include, without limitation, antibody mimics that use non-immunoglobulin protein scaffolds, including adnectins, avimers, aniticalins, single chain polypeptide binding molecules, and antibody-like binding peptidomimetics.

### Antibodies Against Oligomers

In some embodiments, the reagent that specifically binds IAPP oligomers is an antibody. In some embodiments, the antibody is an antibody that binds to toxic IAPP oligomers but not to IAPP monomers or fibrils. In some embodiments, the antibody is A11 antibody or I11 antibody. See, e.g., Kayed et al., Science 300:486-489 (2003); Meier et al., Am. J. Physiol. Endocrinol. Metab. 291:E1317-E1324 (2006); Lin et al., Diabetes 56:1324-1332 (2007), and Gurlo et al., Am. J. Pathol. 176:861-869 (2010), all incorporated herein by reference for all purposes.

Methods for producing polyclonal and monoclonal antibodies that react specifically with a protein of interest are known to those of skill in the art (see, e.g., Coligan, supra; and Harlow and Lane, supra; Stites et al., supra and references cited therein; Goding, supra; and Kohler and Milstein Nature, 256:495-497 (1975)). Such techniques include antibody preparation by selection of antibodies from libraries of recombinant antibodies in phage or similar vectors (see, Huse et al., supra; and Ward et al., supra). For example, in order to produce antisera for use in an immunoassay, the protein of interest or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the sequences disclosed herein and conjugated to a carrier protein can be used as an immunogen.

Polyclonal sera are collected and titered against the immunogen protein in an immunoassay, for example, a solid phase immunoassay with the immunogen immobilized on a solid support. Polyclonal antisera with a titer of 10⁴ or greater are selected and tested for their cross-reactivity against non-IAPP proteins, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

A number of proteins of the invention comprising immunogens may be used to produce antibodies specifically or selectively reactive with the proteins of interest. Recombinant protein is an exemplary immunogen for the production of monoclonal or polyclonal antibodies. Naturally occurring protein may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described supra. The product is then injected into an animal capable of producing antibodies. Either monoclonal or polyclonal antibodies may be generated for subsequent use in immunoassays to measure the protein.

Methods of production of amyloid oligomer-specific antibodies are known to those of skill in the art. See, e.g., Kayed et al., Science 300:486-489 (2003). In brief, a molecular mimic of soluble oligomers is synthesized that mimics the structural organization of Aβ in micellar oligomers by attaching the C-terminus of synthetic Aβ peptides to colloidal gold particles via a thioester bond. The molecular mimics, which are of the same approximate size as the naturally formed oligomeric intermediates and which have the same β-sheet secondary structure and properties as determined by circular dichroism, are then mixed with an adjuvant and animals are immunized. The animal's immune response to the immunogen preparation is monitored by taking test bleeds and determining the titer of reactivity to the soluble oligomers. When appropriately high titers of antibody to the immunogen are obtained, blood is collected from the animal and antisera are prepared. Further fractionation of the antisera to enrich for antibodies reactive to the protein can be done if desired (see, Harlow and Lane, supra). Specificity of the anti-oligomer antibody can be determined by testing for the lack of reactivity of the antibody with monomeric protein or fibrillar deposits.

Once target protein specific antibodies are available, the protein can be measured by a variety of immunoassay methods with qualitative and quantitative results available to the clinician. For a review of immunological and immunoassay procedures in general, see, Stites, supra. Moreover, the immunoassays of the present invention can be performed in any of several configurations, which are reviewed extensively in Maggio Enzyme Immunoassay, CRC Press, Boca Raton, Fla. (1980); Tijssen, supra; and Harlow and Lane, supra.

### Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. The molecules can also be conjugated directly to signal generating compounds, e.g., by conjugation with an enzyme or fluorescent compound. A variety of enzymes and fluorescent compounds can be used with the methods of the present invention and are well-known to those of skill in the art (for a review of various labeling or signal producing systems which may be used, see, e.g., U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Alternatively, simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

### Samples for Detection

Samples for detection of IAPP oligomer may be obtained from any tissue or fluid from a human or non-human animal including, but not limited to, plasma and serum. In some embodiments, the sample is a blood sample. In some embodiments, the sample is heart tissue.

### Reducing the Propensity for Heart or Kidney Failure

In some embodiments, wherein it is determined that an individual has a propensity for heart or kidney failure, the method further comprises designing a treatment plan to reduce the propensity for heart or kidney failure in the individual. In some embodiments, the method further comprises administering at least one medication to the individual that reduces the propensity for heart or kidney failure or heart damage. In some embodiments an individual can prevent heart or kidney failure by reinforcing cell membranes before said membranes are damaged by toxic IAPP oligomers.

The duration of treatment for heart or kidney failure can vary: it may be as short as 3 or 6 months, or may be as long as 18 months, 2 years, 5 years, 10 years, or longer. In some cases, the treatment may last the remainder of a patient's natural life. Effectiveness of the treatment may be assessed during the entire course of administration of the treatment after a certain time period, e.g., every 3 months or every 6 months for an 18-month treatment plan. In other cases, effectiveness may be assessed every 9 or 12 months for a longer treatment course. The administration schedule (dose and frequency) of a treatment may be adjusted accordingly for any subsequent administration. Alternatively, the treatment that is administered (e.g., type of medication) may be adjusted accordingly for any subsequent administration.

In some embodiments, a treatment plan comprises administering one or more medications that relieve or alleviate the symptoms and/or causes of heart or kidney failure. In some embodiments, once there is a determination that the level of IAPP toxic oligomers that are present in a sample, such as blood or heart tissue, at levels higher than normal levels (i.e., levels of IAPP toxic oligomers in control samples), the method further comprises designing a treatment plan for the administration of, and subsequently administering, a treatment that relieves, alleviates, or counteracts the activity of the IAPP toxic oligomers. In some embodiments, the method further comprises designing a treatment plan for the administration of, and subsequently administering, one or more of the following treatments: intravenous delivery of a membrane sealant that can seal damaged sarcolemma and improve calcium cycling or restore calcium cycling back to normal levels in cardiac myocytes; administration of a solubilizer of oligomers; administration of insulin to reduce the demand of insulin and IAPP production on pancreatic β-cells; and administration of one or more insulin sensitizing drugs that increase the uptake of glucose by cells and decrease blood glucose levels. In some embodiments, a membrane sealant comprising a poloxamer such as Poloxamer 188 (P188) is administered. In some embodiments, the insulin that is administered comprises a recombinant human insulin or insulin analog that is rapid-acting, short-acting, intermediate-acting, or long-acting. In some embodiments, the insulin-sensitizing drug that is administered comprises a biguanide (e.g., metformin) or a thiazolidinedione (e.g., troglitazone, rosiglitazone, and pioglitazone).

In some embodiments, the treatment that relieves, alleviates, or counteracts the activity of the IAPP toxic oligomers is provided in combination with another therapeutic agent for relieving or alleviating the causes and/or symptoms of heart or kidney failure, such as an Angiotensin-Converting Enzyme (ACE) inhibitor, an angiotensin receptor blocker, a beta blocker, a diuretic, a positive inotrope, or a vasodilator. Accordingly, in some embodiments, the treatment that relieves, alleviates, or counteracts the activity of the IAPP toxic oligomers is administered to a patient who is also being treated with an ACE inhibitor such as a sulfhydryl-containing ACE inhibitor, e.g., captopril or zofenopril; a dicarboxylate-containing ACE inhibitor, e.g., enalapril, ramipril, quinapril, perindopril, lisinopril, or benazepril; and a phosphonate-containing ACE inhibitor such as fosinopril. In other embodiments, the treatment that relieves, alleviates, or counteracts the activity of the IAPP toxic oligomers is administered to a patient that is being treated with an angiotensin receptor blocker such as candesartan, losartan, irbesartan, valsartan, olmesartan, telmisartan, or eprosartan; or a beta blocker such as bisoprolol, carvedilol, and metoprolol. In some embodiments, the treatment that relieves, alleviates, or counteracts the activity of the IAPP toxic oligomers is administered to a patient who is being treated with a diruretic, such as a loop diuretics (e.g., furosemide, bumetanide); a thiazide diuretics (e.g., hydrochlorothiazide, chlorthalidone, chlorthiazide); a potassium-sparing diuretic (e.g., amiloride); and/or spironolactone or eplerenone. As understood in the art, a patient may be treated with various combinations of such agents in addition to receiving a treatment that relieves, alleviates, or counteracts the activity of the IAPP toxic oligomers.

Treatments to reduce the propensity for heart or kidney failure may be administered in a wide variety of oral, parenteral and topical dosage forms. Thus, the treatments to reduce the propensity for heart or kidney failure can be administered by injection, that is, intravenously, intramuscularly, intracutaneously, subcutaneously, intraduodenally, or intraperitoneally; by inhalation, for example, intranasally; or transdermally.

Methods of Screening for Agents that Reduce the Propensity for Heart or Kidney Failure

In another aspect, the invention provides for a method for screening for agents that prevent or reduce the propensity for heart or kidney failure in an individual who has a diabetic condition, such as type 2 diabetes or pre-diabetes, the method comprising screening a plurality of agents for the ability: to enhance excretion of IAPP oligomers from the body and/or to block or interfere with the formation of IAPP oligomers.

In some embodiments, the method further comprises identifying at least one agent from the plurality that enhances excretion of IAPP oligomers from the body and/or blocks or interferes with the formation of IAPP oligomers; and administering the identified agent to an animal and measuring the ability of the agent to reduce the rate of heart or kidney failure.

### Agents That Reduce the Propensity for Heart or Kidney Failure

The agents that reduce the propensity for heart or kidney failure may comprise agents that enhance the excretion of IAPP oligomers, e.g., by solubilizing the IAPP oligomers, or alternatively, agents that block or interfere with the formation of IAPP toxic oligomers, e.g., by blocking monomers from forming intermediate IAPP oligomers or by blocking intermediate IAPP oligomers from forming toxic oligomers. The agents screened for enhancing the excretion of IAPP oligomers or for blocking or interfering with IAPP oligomer formation can be any small chemical compound, or a biological entity, such as a protein, sugar, nucleic acid or lipid. Typically, test compounds will be small chemical molecules and peptides. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### Methods of Screening

A number of different screening protocols can be utilized to identify agents that enhance the excretion of IAPP oligomers block or interfere with the formation of IAPP toxic oligomers in cells, particularly mammalian cells, and especially human cells. In general terms, the screening methods involve screening a plurality of agents to identify an agent that enhance the excretion of IAPP oligomers by, e.g., breaking down or solubilizing IAPP oligomers, or that block or interfere with the formation of IAPP toxic oligomers by, e.g., binding to an IAPP monomer or an IAPP oligomer.

For screening for agents that enhance the excretion of IAPP oligomers, any cell expressing IAPP oligomers can be used. For screening for agents that block or interfere with the formation of IAPP oligomers, any cell expressing IAPP monomers or oligomers can be used. In some embodiments, the cells are eukaryotic cell lines (e.g., CHO or HEK293) transformed to express IAPP monomers or oligomers. In some embodiments, a cell that endogenously expresses IAPP monomers or oligomers is used in screens.

### Polypeptide Binding Assays

For screening for agents that block or interfere with the formation of IAPP oligomers, preliminary screens can be conducted by screening for agents capable of binding to IAPP monomers or oligomers, as at least some of the agents so identified are likely to block or interfere with the formation of IAPP oligomers. Binding assays are also useful, e.g., for identifying endogenous proteins that interact with IAPP oligomers. For example, antibodies or other molecules that bind IAPP oligomers can be identified in binding assays.

Binding assays usually involve contacting an IAPP monomer or oligomer with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89). Other binding assays involve the use of mass spectrometry or NMR techniques to identify molecules bound to an IAPP monomer or oligomer or displacement of labeled substrates. The IAPP monomers or oligomers utilized in such assays can be naturally expressed, cloned or synthesized.

In mammalian or yeast two-hybrid approaches (see, e.g., Bartel, P. L. et. al. Methods Enzymol, 254:241 (1995)) can be used to identify polypeptides or other molecules that interact or bind when expressed together in a host cell.

### Oligomerization Assay

The effect of an agent on the formation of IAPP oligomers can be screened using an oligomerization assay. As a non-limiting example, a thioflavin T (TFT) fluorescence assay can be used to measure the ability of IAPP to form oligomers. See Lin et al., J. Clin. Endocrinol. Metab. 90:6678-6686 (2005); Meier et al., Am. J. Physiol. Endocrinol. Metab. 291:E1317-E1324 (2006), incorporated herein by reference for all purposes. Briefly, IAPP monomer and the agent to be screened are incubated with thioflavin T, a dye known to preferentially bind amyloid fibrils, and fluorescence is measured at multiple timepoints to measure IAPP oligomerization. Using the TFT assay, if a solution of agent and IAPP monomer exhibited less fluorescence signal than a control solution (e.g., a solution of IAPP monomer alone), then that agent would be identified as blocking or interfering with the formation of IAPP oligomers.

### Oligomer Excretion Assay

The effect of an agent on enhancing the excretion of IAPP oligomers can be screened in vivo, for example by administering an agent to an animal expressing IAPP oligomers and measuring the levels of IAPP oligomers that are excreted from the animal, e.g. in a bodily fluid such as urine. The levels of excreted IAPP oligomers can be measured using an immunoassay as described herein, such as by dot blot or Western blot analysis using anti-IAPP and A11 antibodies. Using such an assay, if the administration of an agent to the animal resulted in increased levels of IAPP excreted by the animal as compared to a control animal (e.g., an animal not administered the agent), then that agent would be identified as enhancing the excretion of IAPP oligomers.

### Two-Step Screen

In some embodiments, the method of screening for agents comprises screening a plurality of agents for the ability to enhance excretion of IAPP oligomers from the body and/or to block or interfere with the formation of IAPP oligomers, and further comprises administering the identified agent to an animal and measuring the ability of the agent to reduce the rate of heart or kidney failure. Agents that are identified by any of the foregoing screening methods can be administered to an animal that serves as a model for human diabetic conditions or human heart or kidney failure, then the ability of the agent to reduce the rate of heart or kidney failure in that animal is measured. For example, if the animal serves as a model for human diabetic conditions, the ability of the agent to reduce the rate of heart or kidney failure in the animal can be measured by any known test for diabetic conditions, such as the HbA1c test, the C-peptide) test, the FPG test, the OGTT test, and/or the casual plasma glucose test. If the animal serves as a model for human heart or kidney failure, the ability of the agent to reduce the rate of heart or kidney failure in the animal can be measured by, for example, echocardiography, MRI, micromanometer conductance catheters, or by measuring calcium transient amplitudes in cardiac myocytes. The animal models utilized in such screens generally are mammals of any kind. Specific examples of suitable animals include, but are not limited to, primates, mice and rats.

### Compositions, Kits, and Integrated Systems

The invention compositions, kits and integrated systems for practicing the methods described herein using IAPP polypeptides of the invention, antibodies, etc.

The invention provides assay compositions for use in solid phase assays; such compositions can include, for example, one or more IAPP polypeptides immobilized on a solid support, and a labeling reagent. In each case, the assay compositions can also include additional reagents that are desirable for hybridization. Modulators of activity of an IAPP polypeptide of the invention can also be included in the assay compositions.

In some embodiments, the solid support comprises a sensor operably linked to one or more nanoparticles, wherein the one or more nanoparticles are conjugated to an antibody that specifically binds IAPP oligomers. As used herein, the term “nanoparticle” refers to a defined particle of typically 5 to 5000, or more typically 5 to 500 atoms. Typically, the nanoparticles have dimensions of less than 150 nanometers. In some embodiments, nanoparticles may be made from such materials as metal, such as silver or gold; semiconductor material; carbon; or biological materials such as nucleic acids or peptides.

The invention also provides kits for predicting the propensity for heart or kidney failure in an individual who has a diabetic condition such as type 2 diabetes or pre-diabetes. The kits typically include a probe which comprises an antibody that specifically binds to oligomers or IAPP oligomers, and a label for detecting the presence of the probe. Kits optionally further include additional components such as instructions to practice a high-throughput method of assaying for an effect on activity of the IAPP oligomers of the invention, one or more containers or compartments (e.g., to hold the probe, labels, or the like), a control modulator of the activity of IAPP oligomers, a robotic armature for mixing kit components or the like.

The invention also provides integrated systems for high-throughput screening of potential modulators for an effect on the activity of an IAPP oligomer of the invention. The systems can include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

The following examples are offered to illustrate, but not to limit, the claimed invention.

### EXAMPLE 1

IAPP Toxic Oligomers are Present in Blood from T2DM Humans

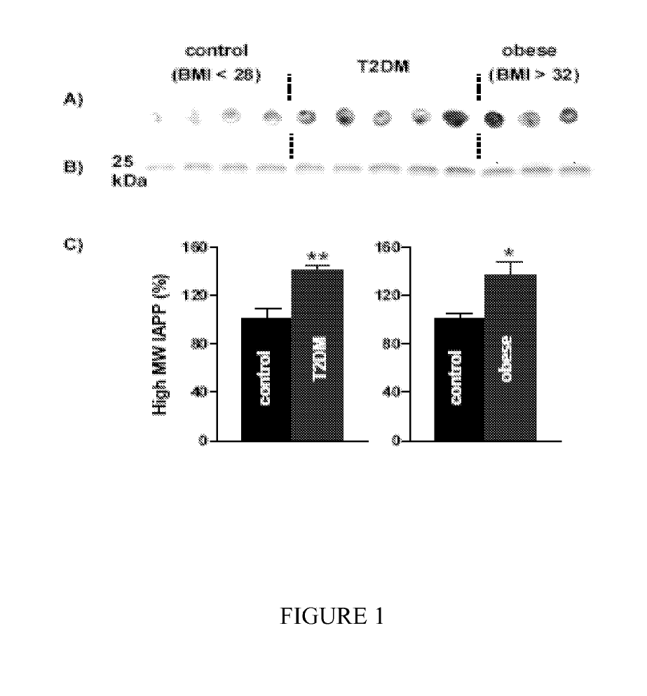

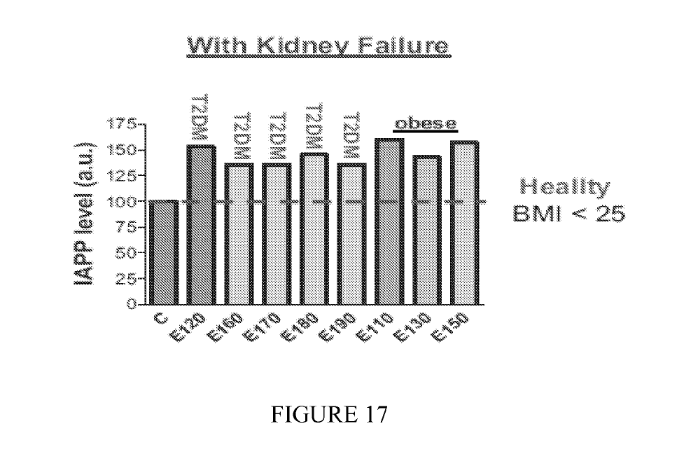

Blood samples were collected from type 2 diabetes mellitus (“T2DM”), overweight (Body Mass Index>25) and lean healthy subjects enrolled in an epidemiological study on cardiovascular diseases at the University of California Davis Medical Center. Using an antibody specific for toxic oligomers (A11), (Kayed et al., Science 300:486-489 (2003)), toxic oligomers in serum samples from T2DM patients and from overweight individuals were detected (FIG. 1A). Oligomer-specific immunoreactivity was significantly abundant in T2DM patients and obese (BMI>32) individuals compared to non-diabetic, non-obese (BMI<28) individuals. This correlates with larger bands corresponding to high molecular weight IAPP species (˜25 kDa and ˜50 kDa) that can be seen on western blots using an anti-IAPP antibody (FIG. 1B). The average anti-IAPP specific immunoreactivity signal derived by the integration of bands at 25 kDa and 50 kDa was about 40% larger for T2DM and obese individuals than for control individuals (FIG. 1C). The average anti-IAPP specific immunoreactivity signal was also about 40% larger for T2DM and obese individuals with kidney failure than for control individuals (FIG. 17).

IAPP Amyloids Accumulate in the Heart in Patients with Obesity and Type-2 Diabetes

Heart specimens were obtained at the time of orthotopic heart transplantation at the Hospital of University of Pennsylvania (for failing hearts) or organ donation (for non-failing hearts) in accordance with the Institutional Review Board approval. Inclusion in tissue-based studies was not restricted on the basis of age, gender, race or ethnic status.

A total of 53 human hearts (left ventricle) were divided in pathologically distinct groups as follows. DM-HF represents the group of failing hearts from patients with overt type-2 diabetes pre-transplantation (N=25). Both ischemic (ICM) and congestive (DCM) failing hearts were included in the study (S1). With few exceptions, patients in this group were either overweight, i.e. 25<BMI<30, (N=7) or obese, i.e. BMI≧30, (N=14), at the date of heart transplant. Some patients in this group were in an advanced stage of diabetes, as they received insulin (N=17). No patient included had a history of ketoacidosis. Other patients in the diabetes group received oral hypoglycemic agents alone (N=6), prior heart transplant. OW/OB-HF stands for failing hearts from overweight/obese patients, i.e. BMI≧25, (N=8). Patients in this group presented severely impaired glucose tolerance in response to steroid exposure and rapid transition (<1 yr) to overt diabetes, posttransplantation. The OW/OB-NF group (N=8) includes non-failing hearts from overweight/obese individuals. Heart samples from lean (L), healthy patients without heart failure, i.e. the L-NF group (N=5), and from lean patients with heart failure but no diabetes, i.e. the L-HF group (N=7), served as controls. The L-HF group corresponds to patients with advanced chronic HF of variable duration (range 0.5 to 8 years) and included both individuals with ischemic and nonischemic etiologies for their HF, as shown in Table 1.

TABLE 1
Heart failure etiology, gender, age, and BMI for all patients from
heart tissues analyzed.

| | HF | | | |
|---|---|---|---|---|
| Code | Etiology | Gender | Age | BMI |
| DM-HF | ICM | M | 50 | 32 |
| (Insulin) | | | 58 | 25.7 |
| | | | 59 | 31.4 |
| | | | 64 | 36.6 |
| | | | 58 | 25.7 |
| | DCM | F | 61 | 28 |
| | | | 44 | 31.2 |
| | | | 43 | 40.3 |
| | | M | 45 | 32.2 |
| | | | 54 | 28.6 |
| | | | 64 | 25.5 |
| | | | 59 | 27.7 |
| | | | 67 | 31.2 |
| | | | 56 | 32.1 |
| | | | 47 | 35.2 |
| DM-HF | ICM | M | 60 | 35 |
| (Hypoglycemics) | | | 66 | 20.6 |
| | | | 56 | 31 |
| | | | 60 | 20.6 |
| | | | 63 | 24.3 |
| | DCM | M | 38 | 32.1 |
| | | | 58 | 34 |
| | | | 66 | 23.1 |
| | | | 58 | 34 |
| | | F | 58 | 28.4 |
| OW/OB-HF | ICM | M | 42 | 28 |
| | | | 58 | 22.7 |
| | | | 63 | 25.2 |
| | | | 54 | 23.8 |
| | | | 47 | 25.9 |
| | | | 63 | 24 |
| | | | 49 | 32.7 |
| | | F | 54 | 29.4 |
| OW/OB-NF | | F | 52 | 24.2 |
| | | | 51 | 28 |
| | | | 60 | 31.2 |
| | | M | 51 | 31.9 |
| | | | 43 | 34.1 |
| | | | 50 | 31.8 |
| | | | 52 | 31.2 |
| | | | 59 | 28.7 |
| L-NF | | F | 37 | 20.8 |
| | | | 33 | 24.3 |
| | | M | 20 | 23.7 |
| | | | 45 | 25.1 |
| | | | 46 | 20.1 |
| L-HF | ICM | M | 48 | 19.7 |
| | | | 25 | 22.1 |
| | | | 58 | 22.7 |
| | DCM | F | 44 | 20.7 |
| | | M | 57 | 23.9 |
| | | | 34 | 19.9 |
| | | | 46 | 21.8 |

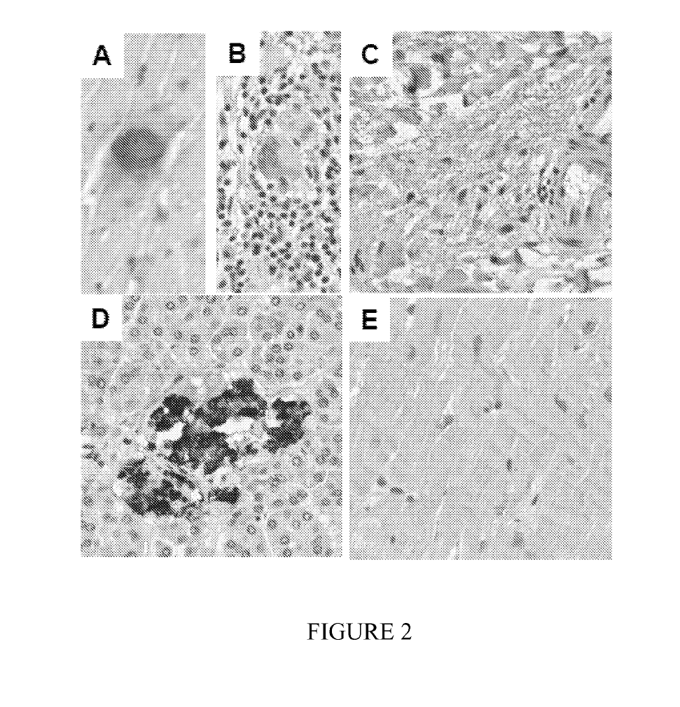

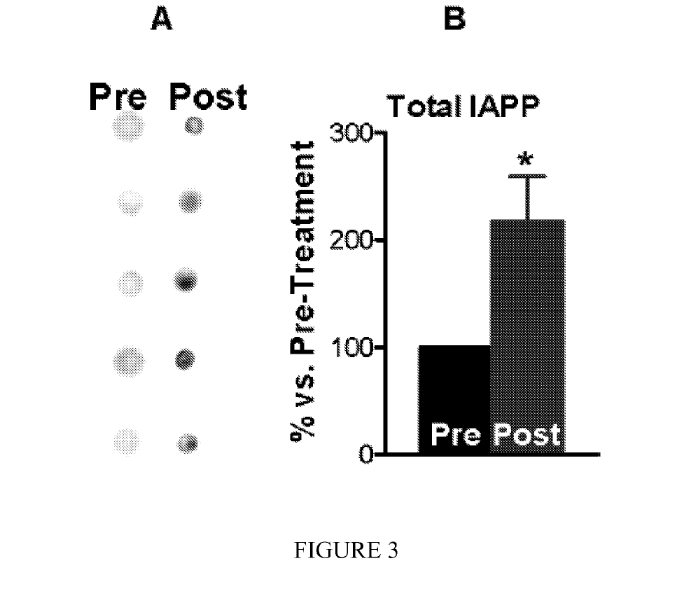

Immunohistochemistry with an anti-IAPP antibody shows large IAPP deposits in failing hearts from diabetic patients (FIG. 2A-C) similar to those in pancreatic islets from diabetic patients (FIG. 2D). IAPP deposits are scattered through the heart and show typical plaque (FIG. 2A, B) and fibrillar tangle (FIG. 2C) type of structures. Typically, IAPP deposits are formed at sites with myocyte multinucleation, variation in nuclear size and infiltrating cells (FIG. 2B,C) which usually occur with fibrotic and infiltrative diseases. In contrast, left ventricle sections from normal hearts (FIG. 2E) do not show IAPP deposition and structural abnormalities. To quantify the IAPP deposition in plaques and fibrils, the amyloids were disaggregated with formic acid and guanidine hydrochloride. Dot blots showed significantly increased IAPP levels in post-treatment-versus pre-treatment samples (FIG. 3A-B). This indicates that large IAPP aggregates fragmented into small oligomers that were recognized by the anti-IAPP antibody.

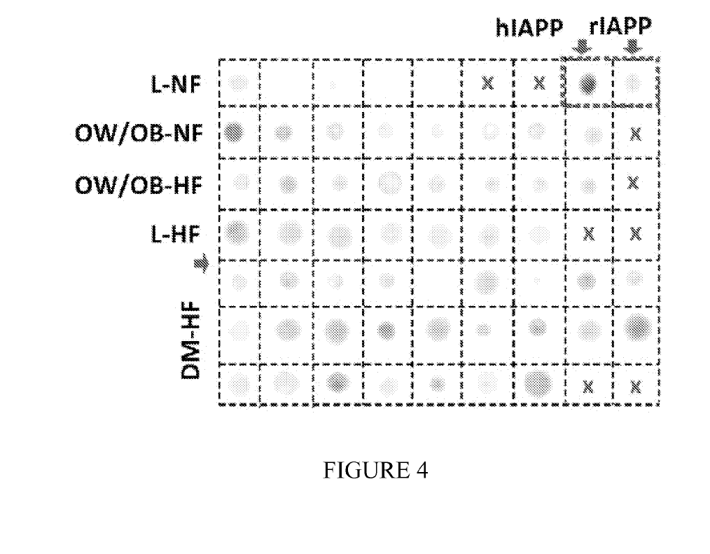

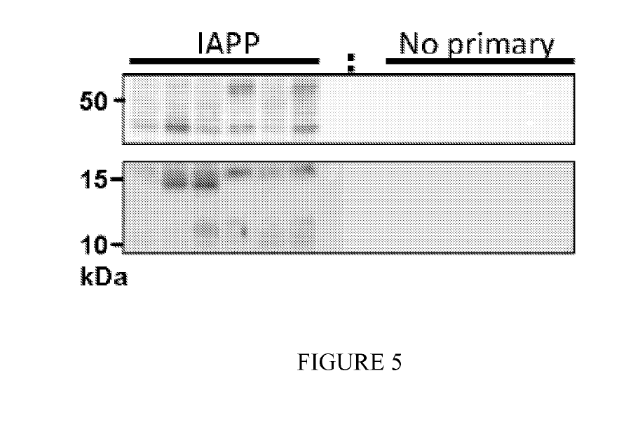

Dot blots with the A-11 antibody, which is specific for toxic oligomers, show the presence of toxic oligomers amyloidogenic entities within the heart in patients with overweight/obesity (OW/OB) and diabetes (DM) (FIG. 4). Non-failing (NF) hearts from lean (L), L-NF group (control), lack toxic oligomers. The percentage of strong A-11 immunoreactivity signals (darker dots) is somewhat larger in hearts from the patients with diabetes, which also correlates with the western blot analysis indicating rich accumulation of IAPP entities with molecular weights greater than ˜16 kDa in these hearts. Apparently, IAPP oligomers are equally present in both ischemic (ICM) and non-ischemic (DCM) hearts (FIG. 4). Hearts in the L-HF group also showed increased A-11 immunoreactivity, which may indicate either undiagnosed metabolic dysfunction or the presence of toxic oligomers of other source. Dots on the upper, right side corner show positive (human IAPP) and negative (rat IAPP) controls for oligomer formation (50 μM recombinant protein incubated at room temperature for 24 hours) (FIG. 4). Before the test with A-11 antibody, all protein homogenate samples were incubated with Protein A-coated magnetic beads to extract IgG, a possible source of cross-reactivity. A test of efficiency of IgG removal is shown in FIG. 5.

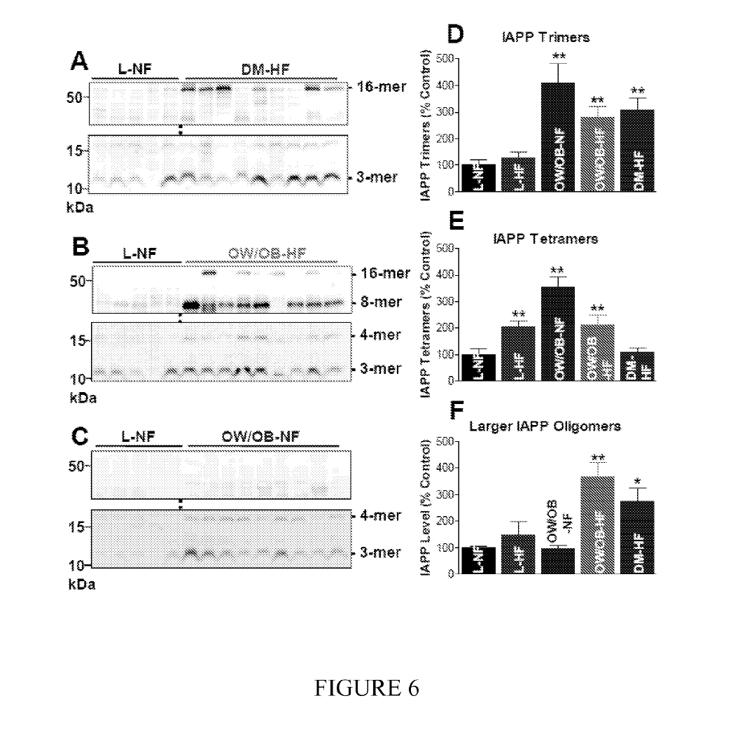

To assess the levels and characteristic size distributions of soluble IAPP oligomers accumulated in left ventricles, western blots with an anti-IAPP antibody on left ventricle protein homogenates was performed. Molecular weight bands correspond to IAPP trimers (12 kDa), tetramers (16 kDa) and two additional larger molecular weight structures at ˜32 kDa (octamers) and ˜64 kDa (16-mers) (FIG. 6A-C). Negative controls indicated that these bands are specific (FIG. 5). Intensity signal analysis (FIG. 6D-F) indicated that cardiac IAPP oligomer accumulation is markedly larger in failing hearts from patients with type-2 diabetes and overweight/obesity than in normal hearts and failing hearts from patients without diabetes (controls). Intriguingly, large IAPP oligomers, i.e. >32 kDa, are abundant in failing hearts from diabetic and obese patients (FIG. 6A,B,F), but not in non-failing hearts from overweight/obese individuals (FIG. 6C,F). In contrast, smaller IAPP oligomers were already elevated in non-failing hearts from overweight/obese patients (FIG. 6C-E), indicating an early stage of IAPP buildup in the heart. These results imply that the size of IAPP oligomers accumulating in the heart may be critical in inducing deleterious cardiac effects. IAPP tetramers were also present to some extent in failing hearts from non-diabetic patients (FIG. 6E), which might indicate undiagnosed insulin resistance in those patients, a normal occurrence in ageing. Generally, amyloid oligomers in the size range found in failing hearts (FIG. 6A-C) demonstrate increased toxicity in various other types of cells and tissues. They are recognized by the A-11 oligomer-specific antibody. Dot blots with A-11 antibody (FIG. 4) indicate the presence of toxic oligomers in hearts from overweight/obese and diabetic patients and in failing hearts from lean patients, which is in agreement with western blot data (FIG. 6). In contrast, normal hearts from lean humans lack toxic oligomers.

IAPP Amyloids Accumulate in the Kidney in Patients with Obesity and Type-2 Diabetes

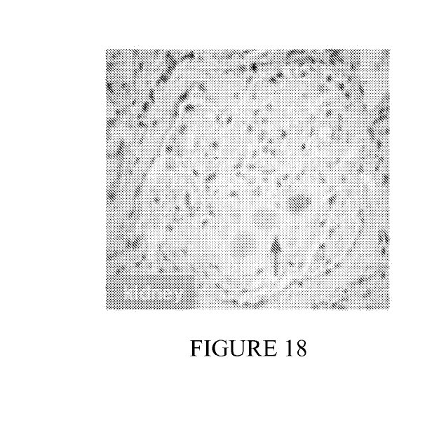

Immunohistochemistry with an anti-IAPP antibody shows large IAPP deposits in kidneys from diabetic patients (FIG. 18) similar to those in pancreatic islets from diabetic patients (FIG. 2D). IAPP deposits are scattered through the kidney and show typical plaque structures (FIG. 18).

### Accumulation of IAPP Toxic Oligomers in the Heart in HIP Rats

To confirm accumulation of IAPP toxic oligomers in the heart in T2DM, HIP rats were used, which overexpress human IAPP in pancreatic β-cells. The HIP rat has been well characterized with respect to IAPP toxic oligomer formation in pancreatic islets (Dobson C M. Trends Biochem. Sci. 24:329-332 (1999) and it has been shown that humans with T2DM and HIP rats share in common the formation of IAPP toxic oligomers in the secretory track of pancreatic β-cells, deficit in β-cell mass and islet amyloid (Gurlo T. et al. Am J Pathol. 176(2):861-9 (2010); Huang C. J. et al., J Biol Chem. 285:339-48 (2010)).

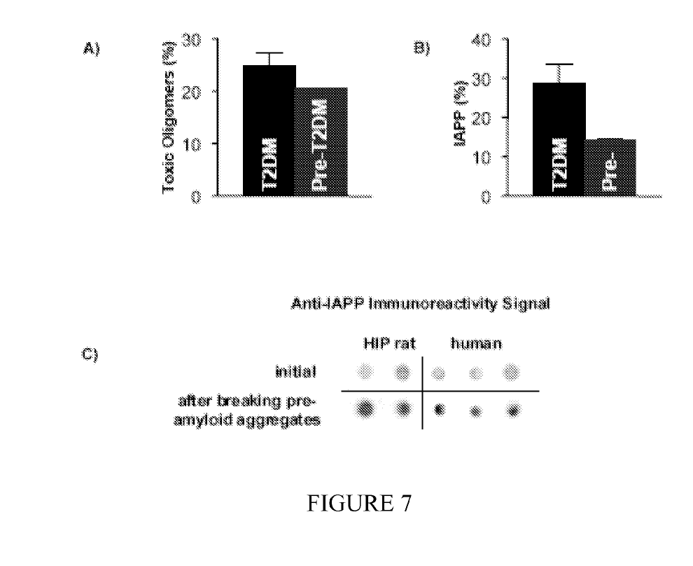

As shown in FIG. 7, HIP rats also demonstrate an accumulation of toxic oligomers in the heart (FIG. 7A) that correlates with the overall increase of IAPP cardiac content (FIG. 7). Oligomer immunoreactivity was more abundant in diabetic subjects than in pre-diabetic subjects, which could just reflect a difference in age and/or disease development. However, the data clearly demonstrate accumulation of toxic oligomers in the heart starting from the early pre-diabetes stage.

To clarify the presence of IAPP in toxic preamyloid entities detected in hearts from T2DM humans and HIP rats, protein homogenates were treated with formic acid, freeze dried and the resulting powders resuspended in guanidine hydrochloride to disaggregate the amyloidogenic entities. By using the anti-IAPP antibody on dot blots, we detected significantly increased IAPP levels in treated versus nontreated samples (see FIG. 7), suggesting that large amyloidogenic structures fragmented into much smaller entities that are recognized by the anti-IAPP antibody.

### Cardiac IAPP Accumulation Alters Ca²⁺ Cycling in Myocytes

Intracellular Ca²⁺ ([Ca²⁺]ᵢ) is central to cardiac myocyte contractility and viability (Netticadan T. et al., Diabetes 50:2133-8 (2001)) and [Ca²⁺]ᵢ dysregulation plays an important role in the pathophysiology of heart disease (Netticadan T. et al., Diabetes 50:2133-8 (2001)), including diabetic cardiomyopathy (Pereira L. et al., Diabetes 55:608-15 (2006); Netticadan T. et al., Diabetes 50:2133-8 (2001)). Altered [Ca₂₊]ᵢ has also become a major focus in the study of pathogenesis of amyloid-related diseases (Haass C. et al, Nat. Rev. Mol. Cell Biol. 8:101-112 (2007); Huang C J et al., J Biol Chem. 285:339-48 (2010); Kawahara M. et al., J Biol Chem 275:14077-14083 (2000)). In Alzheimer's disease, β-amyloid oligomers induce neuron dysfunction and death through a mechanism involving increased [Ca²⁺]ᵢ (Haass C. et al, Nat. Rev. Mol. Cell Biol. 8:101-112 (2007); Kawahara M. et al., J Biol Chem 275:14077-14083 (2000)). Human IAPP oligomers elevate [Ca²⁺]ᵢ levels in pancreatic β-cells, which triggers cell death by apoptosis (Huang C J et al., J Biol Chem. 285:339-48 (2010)). Thus, it was tested to determine whether IAPP oligomers cause [Ca²⁺]ᵢ mishandling in cardiac myocytes.

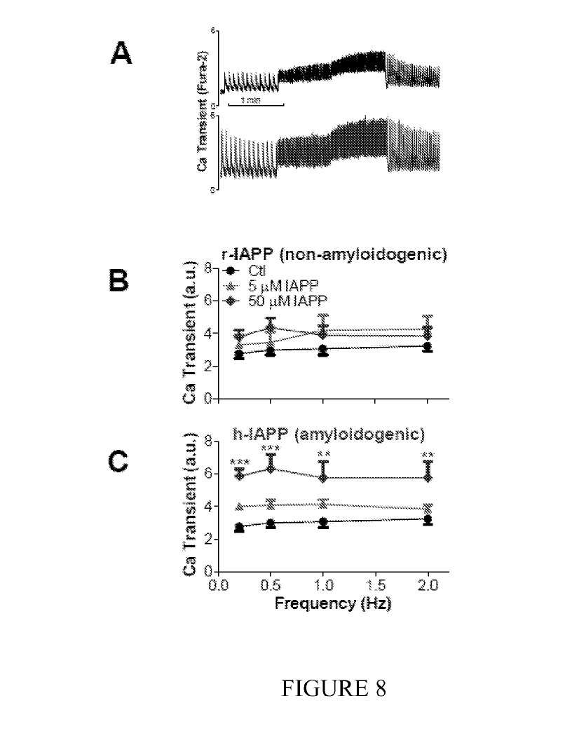

Rat cardiac myocytes were incubated with exogenous human (amyloidogenic) and rat (non-amyloidogenic) IAPP and measured Ca2+ transients produced by field-stimulation at various frequencies (FIG. 8A). Human IAPP, at a concentration (50 μM) at which it rapidly forms oligomers, significantly increased Ca2+ transient amplitude at all stimulation frequencies (FIG. 8C). In contrast, at a similar concentration the non-amyloidogenic rat IAPP induced only a modest, not significant effect (FIG. 8B). These data indicate that IAPP oligomers raise cellular Ca²⁺ load in cardiac myocytes, effect generated also at the interaction with neurons (Kawahara M. et al., J Biol Chem 275:14077-14083 (2000)) and pancreatic β-cells (Huang C J et al., J Biol Chem. 285:339-48 (2010)).

It was then investigated whether in vivo cardiac IAPP accumulation affects Ca²⁺ cycling in a rat animal model of type-2 diabetes. Because rodent IAPP is not amyloidogenic and rodents do not accumulate IAPP amyloids, most rodent models are not adequate for this study. Sprague-Dawley rats transgenic for human IAPP were used in the pancreatic β-cells (HIP rats) (Matveyenko A. and Butler P. C., ILAR Journal, 47:225-233(2006)). These rats show IAPP amyloid deposits in pancreatic islets and gradual decline in β-cell mass leading to impaired fasting glucose at 5 months of age and diabetes by 10 months of age (Matveyenko A. and Butler P. C., ILAR Journal, 47:225-233(2006)). Diabetic rats expressing only the native, non-amyloidogenic rat IAPP isoform (UCD-T2DM rats) (Cummings B. P. et al, Am J Physiol Regul Integr Comp Physiol. 295:R1782-1793 (2008)) were used as negative controls. The UCD-T2DM rats develop diabetes on a time scale similar to HIP rats. Experiments were done at a state of disease development when IAPP secretion is maximal, i.e. in the pre-diabetic stage (Enoki S. et al., Diabetes Res Clin Pract. 15:97-102 (1992); Hayden M R and Tyagi S C, JOP 3:86-108 (2002)), when both glucose and insulin levels in the blood are increased (hyperinsulinemia). Using rats in the pre-diabetic state has also the advantage that one can dissociate the effect of cardiac IAPP accumulation from other confounding factors that affect cardiac Ca²⁺ cycling during late diabetes (Pereira L. et al., Diabetes 55:608-15 (2006); Netticadan T. et al., Diabetes 50:2133-8 (2001)). Indeed, the state of late diabetes is associated with major cardiac remodeling including reduced Ca²⁺ transients and sarcoplasmic reticulum (SR) Ca²⁺ content (Pereira L. et al., Diabetes 55:608-15 (2006); Netticadan T. et al., Diabetes 50:2133-8 (2001)), due to impaired glucose and lipid homeostasis in combination with vascular factors (Guha A. et al., Curr Opin Cardiol. 23:241-8 (2008); Biddinger S. B. and Kahn C. R., Annu. Rev. Physiol. 68, 123-58 (2006); Reaven G. M., J. Clin. Hypertens. 13, 238-243 (2001); Szczepaniak L. S. et al., Circ. Res. 101, 759-67 (2007); Battiprolu P. K., Drug Discov Today Dis Mech 7, e135-e143 (2010); Boudina S. and Abel E. D., Rev. Endocr. Metab. Disord. 11, 31-39 (2010)).

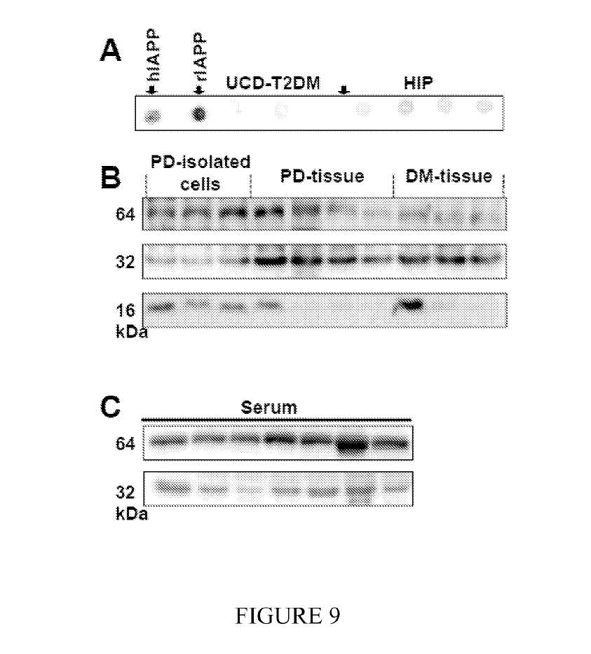

Both HIP (Matveyenko A. and Butler P. C., ILAR Journal, 47:225-233(2006)) and UCD-T2DM (Cummings B. P. et al, Am J Physiol Regul Integr Comp Physiol. 295:R1782-1793 (2008)) rats show ˜2 fold increase in fasting plasma insulin and IAPP levels in pre-diabetes, which is similar to humans with insulin resistance/pre-diabetes (Enoki S. et al., Diabetes Res Clin Pract. 15:97-102 (1992); Hayden M R and Tyagi S C, JOP 3:86-108 (2002), Johnson K. H. et al., Am. J. Pathol. 135, 245-250 (1989); Johnson K. H. et al., N Engl J Med 321, 513-518 (1989); Hayden M. R. JOP 6, 287-302 (2005)). However, IAPP significantly accumulates only in HIP rat hearts (FIG. 9A), a consequence of human IAPP's amyloidogenicity. Western blots on heart protein homogenates, cardiac myocyte lysates and blood serum from HIP rats (FIG. 9B,C) show IAPP molecular weight bands that match those detected in humans (FIG. 6). In HIP rats, the IAPP oligomers circulate through the blood and start accumulating in the heart already in the pre-diabetic state (FIG. 9B,C). Most likely, they attach to sarcolemma or enter the myocyte, as suggested by their presence in cardiac myocyte lysates (FIG. 9B).

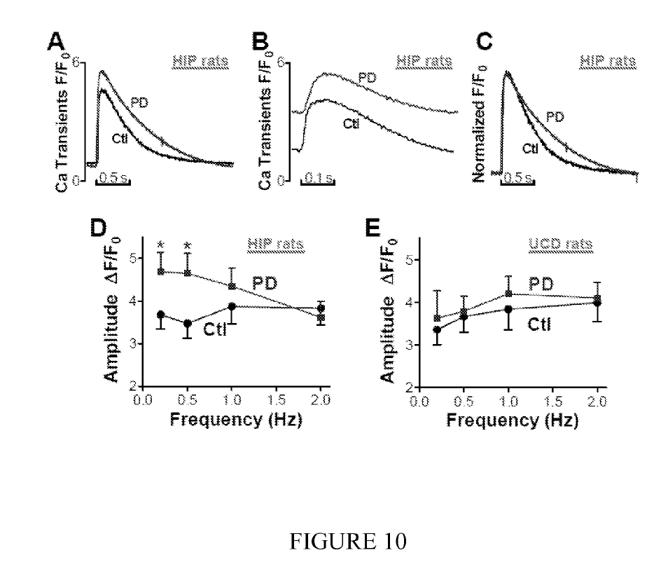

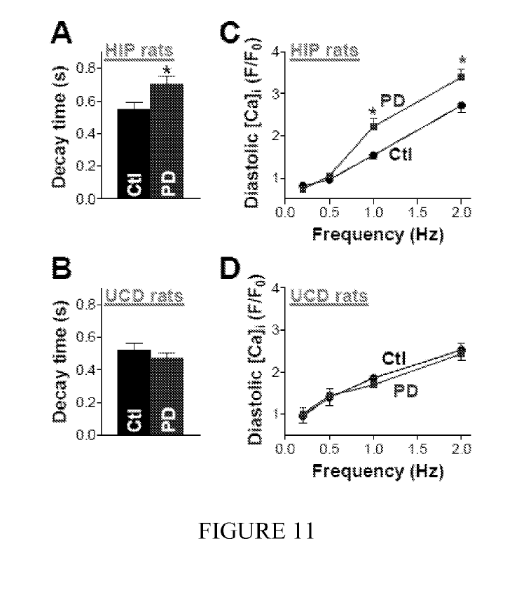

Accumulation of human IAPP oligomers in the heart alters Ca²⁺ cycling in cardiac myocytes from pre-diabetic HIP rats (FIG. 10). At low stimulation frequencies, Ca transient amplitude is significantly larger (4.7±0.5 vs. 3.5±0.3 at 0.5 Hz) in myocytes from pre-diabetic HIP rats versus age-matched control non-diabetic rats (FIGS. 10A,D). In contrast, myocytes from pre-diabetic UCD-T2DM rats show no change in Ca2+ transient amplitude (FIG. 10E). These data suggest that IAPP accumulation in pre-diabetic HIP rats causes the increase in Ca²⁺ transient amplitude, in agreement with our results using exogenous human IAPP oligomers (FIG. 10). Different from age-matched control, Ca²⁺ transient amplitude decreases with increasing the stimulation frequency in myocytes from pre-diabetic HIP rats (negative staircase), so that at 2 Hz the amplitude is similar to that recorded in control rats (FIG. 10B,D). This is probably due to deficiencies in Ca²⁺ re-uptake into the SR. Indeed, Ca²⁺ transient decline, which is mostly due to SR Ca²⁺ re-uptake via the SR Ca-ATPase (SERCA), is significantly slower in pre-diabetic HIP rats vs. control (τ=0.71±0.07 vs. 0.55±0.04 s at a stimulation rate of 0.5 Hz; FIGS. 10C, 11A). In contrast, Ca²⁺ transient decay remains unchanged in myocytes from pre-diabetic UCD-T2DM rats (FIG. 11B). The slower Ca²⁺ transient relaxation in pre-diabetic HIP rats results in an elevated diastolic [Ca²⁺]ᵢ level at higher pacing rates (FIGS. 10B, 11C). Diastolic [Ca²⁺]ᵢ is unaltered in pre-diabetic UCD-T2DM rats (FIG. 11D). Thus, cardiac IAPP oligomer accumulation may accelerate the occurrence of heart dysfunction, and particularly diastolic dysfunction, a typical sign of diabetic cardiomyopathy (Pereira L. et al., Diabetes 55:608-15 (2006); Netticadan T. et al., Diabetes 50:2133-8 (2001); Szczepaniak L. S. et al. Circ. Res. 101, 759-67 (2007); P. K. Battiprolu et al., Drug Discov Today Dis Mech 7, e135-e143 (2010); Boudina S. and Abel E. D., Rev. Endocr. Metab. Disord 11, 31-39 (2010)).

### Cardiac IAPP Accumulation Accelerates Myocyte Remodeling and Hypertrophy

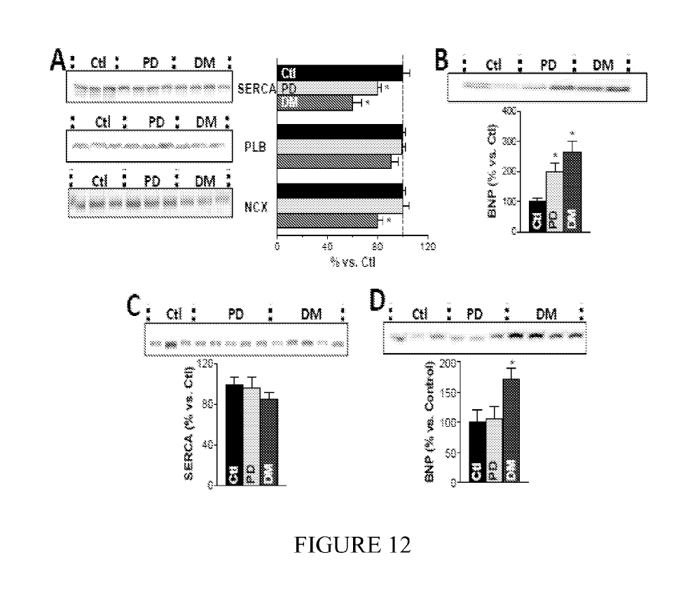

Elevated [Ca²⁺]ᵢ is involved in transcriptional regulation and hypertrophic signaling in the heart (Bers D M., Annu Rev Physiol. 70:23-49 (2008)). The increased cellular Ca²⁺ load in pre-diabetic HIP rats may activate Ca²⁺-dependent transcription pathways, which may alter the transcription of key Ca²⁺ transport and regulatory proteins (Hill and Olson, N Engl J Med 358:1370-80 (2008)). SERCA expression is reduced by 20% and 30% in pre-diabetic and diabetic HIP rats, respectively (FIG. 12A), which may cause the slower Ca²⁺ transient relaxation noted above in pre-diabetic HIP rats. In contrast, SERCA expression was unchanged in pre-diabetic UCD-T2DM rats (FIG. 12C). The protein expression of phospholamban, the endogenous SERCA inhibitor, and Na/Ca exchanger, the main pathway for Ca²⁺ extrusion in cardiac myocytes, are unaltered in pre-diabetic HIP rats (FIG. 12A).

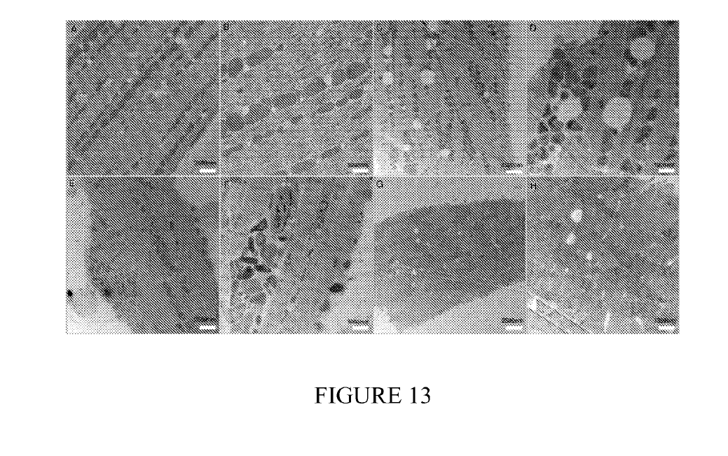

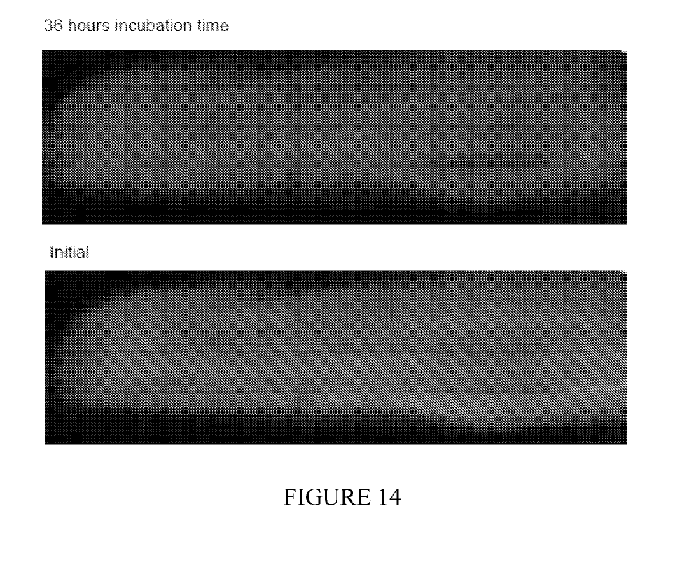

The level of brain natriuretic peptide (BNP), a molecular marker of hypertrophy, is elevated (by 100±30%) in hearts from pre-diabetic HIP rats vs. age-matched control littermates and further increased with diabetes development (FIG. 12B). This result suggests that in HIP rats cardiac hypertrophy begins already in the pre-diabetic stage. In contrast, the BNP level is not altered in pre-diabetic UCD-T2DM rats and only increases after the full development of diabetes (FIG. 12B). Of note, a previous study found that external human IAPP induces hyperthrophy in isolated rat cardiac myocytes (Bell D. et al., J Mol Cell Cardiol 27, 2433-2443 (1995)). Additionally, Cryo-electron microscopy images reveal significant structural modification of the sarcolemma in cardiac myocytes incubated with exogenous IAPP oligomers (FIG. 13). Intriguingly, incubation of isolated rat cardiac myocytes with exogenous IAPP oligomers for 36 hours does not reveal release of calcein from the cytoplasm (FIG. 14). This may indicate that possible pores created in the sarcolemma may be smaller in size than the calcein molecule or significant sarcolemmal damage may require a longer time of incubation. Together, these data suggest that the initial IAPP-mediated increase in Ca²⁺ transient amplitude in pre-diabetic HIP rats may activate Ca²⁺-dependent pathogenic signaling pathways, which exacerbate the pathological gene expression and heart remodeling.

### Reversal of IAPP Oligomer-Induced Membrane Damage

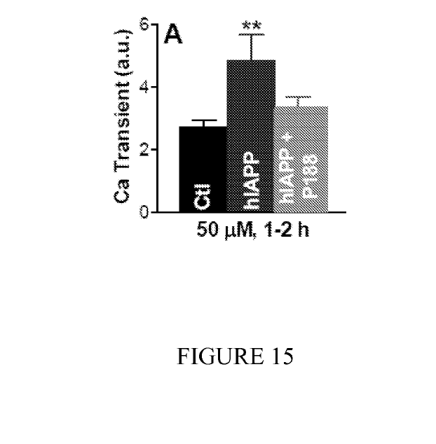

Poloxamer 188 is efficient in sealing damaged membranes (Collins J. M. et al., Biochim. Biophys. Acta 1768, 1238-1246 (2007)) and has been shown to seal damaged neurons incubated with exogenous Aβ oligomers (Mina E. W., J Mol Biol 391, 577-585 (2009)). Oligomerization of Aβ peptides is associated with Alzheimer's disease, and Aβ and IAPP have the same molecular weight sizes and are about 40% identical at the amino acid level. Incubation of cardiac myocytes simultaneously with exogenous IAPP oligomers and poloxamer 188 (1:1 ratio, 50 μM final concentration) reduced the level of alteration of Ca cycling (FIG. 15). This suggests that the polymer molecules either prevent the attachment of IAPP oligomers to sarcolemma or efficiently sealed damaged membranes. Atomic force microscopy approaches demonstrate that IAPP oligomers intercalate into sarcolemma and induce sarcolemma thinning.

### Discussion

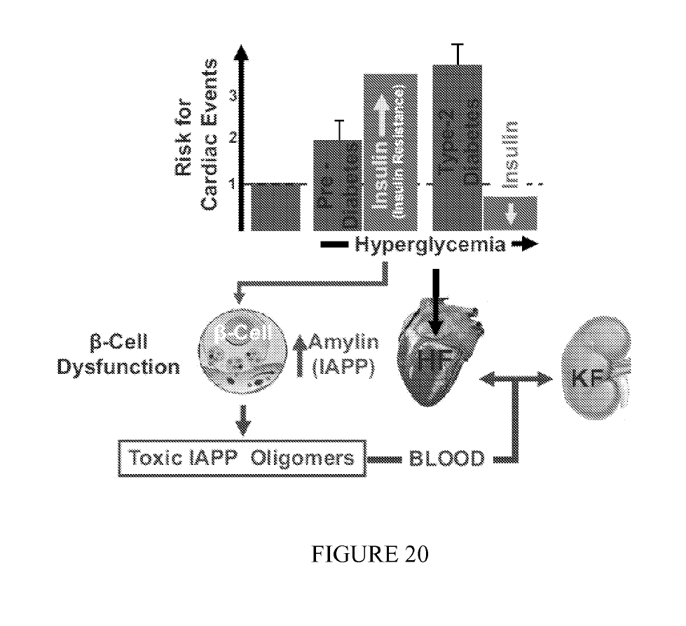

T2DM is associated with a marked increase in cardiovascular (CV) disease (>50% of morbidity and mortality in diabetics) and with poorer outcomes after CV events. T2DM is also linked to cardiomyopathy [20-26], independent of coronary artery disease and hypertension. This clinical entity is characterized by left ventricular hypertrophy, diastolic dysfunction, impaired Ca handling, decreased cardiac efficiency, impaired mitochondrial energetics, increased myocardial lipid storage and inflammation (reviewed in [20-26]). Factors, molecular mechanisms, and time courses underlying diabetic cardiomyopathy are poorly understood [20-26]. It is assumed that the shortage of blood insulin leads to impaired glucose and lipid homeostasis in the heart and transition to heart failure [20-26] (FIG. 20). Epidemiological studies have revealed, however, that severe heart failure often precedes full blown T2DM [27-34], when the blood insulin level is actually elevated (hyperinsulinemia). This clinical state, known as insulin resistance [11,12] increases the risk of heart failure [28]; however, there is no evidence that insulin resistance alone actually causes heart failure [35,36]. T2DM patients with ischemic hearts show surprisingly good myocardial insulin responsiveness [37,38]. It is assumed that conditions secondary to insulin resistance may be causally implicated in cardiac dysfunction. Such conditions may be related to accumulation of IAPP toxic oligomers in the heart. IAPP accumulation in the heart is highly favored in pre-diabetes, as blood hyperinsulinemia is normally accompanied by an increased blood level of the co-secreted IAPP [1, 2, 4]. Significant amounts of IAPP oligomers were found in the blood and in failing hearts from pre-diabetic individuals (FIGS. 1 and 3). Low levels of toxic preamyloid oligomers (other than IAPP) in the heart have been reported to induce cardiomyocyte death and heart failure in mice [8]. Studies have found that IAPP oligomers have toxic effects on cells [2, 5, 9, 10, 13, 14], including cardiomyocytes. Similar amyloidogenic entities, i.e. Aβ oligomers, the molecular entities implicated in the development of Alzheimer's disease, are also extremely toxic [3, 6, 7]. Data show that even nanomolar concentrations of preamyloid oligomers are able to kill mature neurons [39].

The IAPP oligomer is a biomarker of diabetic heart or kidney failure that circulates through the blood and manifests in the heart starting from the early pre-diabetes stage. In pre-diabetic HIP rats, Ca transient amplitudes are significantly increased, which may be the intrinsic signature of the IAPP's amyloidogenity (e.g., pre-diabetic rats bearing only non-amyloidogenic IAPP variant lack this pathology). Intracellular Ca upsurge has also been implicated as a mediator of toxic oligomer-induced cell death and dysfunction in neurodegenerative diseases, such as Alzheimer's disease [15-18]. However, the underlying mechanisms are not fully elucidated [15-18]. Our finding here, that intracellular Ca is increased in pre-diabetes, poses new questions regarding the molecular mechanisms contributing to cardiac dysfunction in T2DM.

Ca cycling is essential for cardiac myocyte contraction and relaxation [19]. Upon depolarization during the action potential, Ca enters the myocytes via voltage-gated L-type Ca channels and induces further Ca release from the SR by activating the SR Ca release channels (or ryanodine receptors; RyRs). This raises the free intracellular Ca level, allowing Ca to bind to the myofilaments and trigger contraction. Relaxation occurs when Ca is removed from the cytosol, mainly by SERCA, which takes Ca back into the SR, and the sarcolemmal NCX. SERCA activity is modulated by its inhibitor PLB. Phosphorylation of PLB relieves the inhibition, and promotes cardiomyocyte relaxation by increasing the Ca affinity of SERCA. Alterations in the function and/or protein expression of all these proteins have been reported in various T2DM rodent models [40-43]. However, such studies only investigated Ca cycling when full-blown T2DM was already present and in the absence of amyloidogenic IAPP (since rat IAPP is non-amyloidogenic). Here, in contrast, we showed that accumulation of IAPP toxic oligomers at the sarcolemma increases Ca influx, leading to larger Ca transients in pre-diabetic HIP rats. This may activate downstream signaling pathways and lead to remodeling that alters the function and/or expression of cardiac Ca cycling proteins.

Alterations in cytosolic Ca may also trigger mitochondrial dysfunction [24, 44], hence affecting cardiac metabolism. Changes in mitochondrial Ca dynamically regulate respiration and can contribute to mitochondrial dysfunction and cell death. While the exact relationship between cytosolic Ca transients and mitochondrial Ca level is controversial [45], it is agreed that lower Ca transients in the cytosol result in lower mitochondrial Ca. This may result in reduced Ca-sensitive dehydrogenases activity and therefore impaired ATP production [44]. Impaired ATP synthesis and reduced cytosolic Ca transients may both contribute to the development of contractile dysfunction in late T2DM. However, the initial consequence of IAPP accumulation in the heart we detected was an increase in Ca transient amplitude (FIG. 8), which may lead to elevated mitochondrial Ca levels. Mitochondrial Ca overload can lead to mitochondrial dysfunction and cell death [44]. Other stressors, such as an increased production of reactive oxygen species (ROS), also affect mitochondrial function in diabetes (reviewed in [44]). Brownlee and co-workers [46, 47] showed that mitochondrial ROS activate pathological pathways that induce diabetic complications. Oxidative stress and increased production of H₂O₂ were demonstrated to contribute to mitochondrial dysfunction in the diabetic stage, via mitochondrial uncoupling mechanisms [44].

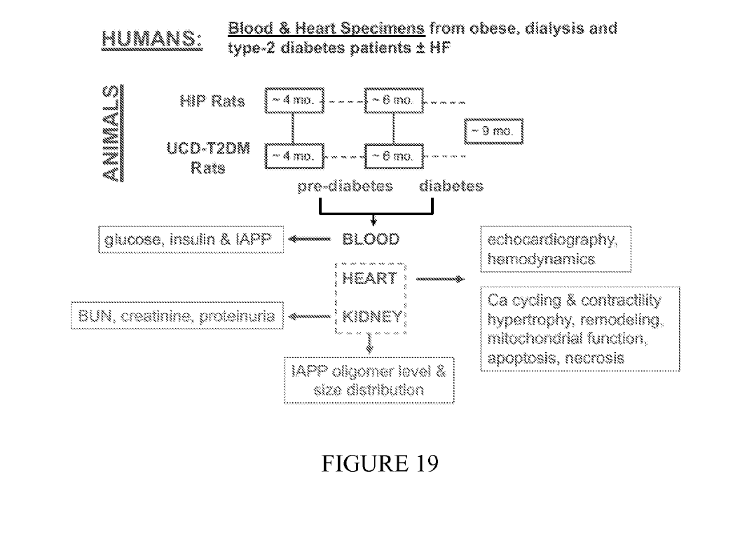

IAPP aggregates are implicated in the occurrence of additional T2DM complications. They participate in stimulating lipolysis, in elevating plasma free fatty acid level, in stimulating advanced glycosylation end-products receptors, in activating the rennin-angiotensin-aldosterone system and in promoting the inflammatory process [48, 49]. Recent epidemiological studies revealed that drugs that stimulate β-cells to produce more insulin (and IAPP) increase the risk of heart failure [50, 51]. Although complications of T2DM are difficult to predict, it is increasingly clear that β-cell dysfunction and formation of IAPP oligomers result in a feed-forward process, whereby the secretion of these amyloidogenic entities in the blood causes additional damage in organs other than pancreas, including the heart (FIG. 19). Thus, IAPP oligomers, which are secondary products to an increased demand for insulin biosynthesis, could be pathogens of diabetic cardiac dysfunction. IAPP mediated cardiotoxicity had remained unnoticed to date because 1) poor prognosis of cardiac dysfunction in T2DM; 2) IAPP's amyloidogenity was considered to manifest only in pancreas [52]; and 3) rodent models (mostly used in diabetic cardiomyopathy studies) bear only the non-amyloidogenic rat IAPP that does not form amyloids.

Promising therapies against IAPP oligomer toxicity may derive from, but is not limited to, the use of polymer molecules to either prevent the attachment of IAPP oligomers to sarcolemma or to efficiently seal damaged membranes. Results described herein show that P188, for example, can attach to patches of thinner sarcolemma and provide protection to myocytes against the IAPP oligomer-induced membrane damage.

In summary, the present results suggest that IAPP oligomers contribute to cardiac dysfunction, independently of hyperglycemia. As they circulate through the blood, these toxic entities may represent an effective target for diagnostic purposes and therapeutic strategies.

### Methods

The animal protocol was approved by IACUC of UC Davis. The weight, glucose, insulin and IAPP levels in the blood were measured monthly both in HIP and wild type rats, as described previously [5]. Generally, glucose, insulin and IAPP levels were in the margins reported previously. Pre-diabetes was defined as the state characterized by (non-fasting) blood glucose and insulin levels greater than normal (>150 mg/dl for glucose; >2 ng/ml for insulin) obtained on two consecutive measurements. When the non-fasting blood glucose level remained higher than normal, but the insulin level decreased on two consecutive measurements, the rat was considered diabetic. Pre-diabetic (150 mg/dl<blood glucose<180 mg/dl) and diabetic (blood glucose>250 mg/dl) rats were euthanized by exsanguinations following excision of the heart quickly.

To elucidate the timeline of accumulation of IAPP toxic oligomers in the heart, a longitudinal study on HIP rats was carried out. The weight, glucose, insulin and IAPP levels in the blood were measured monthly both in HIP and wild type rats, as described previously [5]. Generally, glucose, insulin and IAPP levels were in the margins reported previously.

In each test, we have also included two positive controls (+). An additional strip was used as a negative control, meaning that the samples were blocked and incubated with secondary antibodies only, without incubation with primary antibodies, in order to rule out eventual artifacts (such as sample cross reactivity with secondary antibodies due to the presence of human IgGs in the homogenates).

Heart protein homogenates (30 μg/lane) were separated by SDS-PAGE (15% gels) and blotted onto nitrocellulose membranes. After blocking with 8% nonfat dried milk, membranes were probed with an anti-IAPP primary antibody (from Abcam). We verified the specificity of high molecular bands in western blots by pre-adsorbing the anti-IAPP antibody with purified IAPP. The supernatant was blotted against GAPDH as an input control. All antibodies that were used are commercially available.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Höppener, J. W. M., Ahren, B., and Lips, C. J. M. (2000) Islet amyloid and type 2 diabetes mellitus. N. Engl. J. Med. 343: 411-419
2. Haataja L, Gurlo T, Huang C J, Butler P C. Islet amyloid in type 2 diabetes, and the toxic oligomer hypothesis. Endocr Rev. 2008 May; 29(3):303-16.
3. Selkoe, D. J. (2003) Folding proteins in fatal ways, Nature 426:900-904
4. Young A A (1997) Amylin's physiology and its role in diabetes. Curr Opin Endocrinol Diabetes 4:282-90.
5. Butler A E, Janson J, Bonner-Weir S, Ritzel R, Rizza R A, Butler PC. Beta-cell deficit and increased beta-cell apoptosis in humans with type 2 diabetes. Diabetes. 2003 January; 52(1):102-10.
6. Haass C, Selkoe D J. Soluble protein oligomers in neurodegeneration: lessons from the Alzheimer's amyloid beta-peptide. Nat. Rev. Mol. Cell Biol. 2007; 8:101-112.
7. Kayed R, Head E, Thompson J L, McIntire T M, Milton S C, Cotman C W, Glabe C G. (2003) Common Structure of Soluble Amyloid Oligomers Implies Common Mechanism of Pathogenesis. Science 300:486-489
8. Pattison J S, Sanbe A, Maloyan A, Osinska H, Klevitsky R, Robbins J (2008) Cardiomyocyte Expression of a Polyglutamine Preamyloid Oligomer Causes Heart Failure Circulation 117:2743-2751.
9. Meier J J, Kayed R, Lin C Y, Gurlo T, Haataja L, Jayasinghe S, Langen R, Glabe C G, Butler P C. Inhibition of hIAPP fibril formation does not prevent beta-cell death: Evidence for distinct actions of oligomers and fibrils of hIAPP. Am J Physiol. 2006; 291:E1317-E1324.
10. Gurlo T, Ryazantsev S, Huang C J, Yeh M W, Reber H A, Hines O J, O'Brien T D, Glabe C G, Butler P C. Evidence for proteotoxicity in beta cells in type 2 diabetes: toxic islet amyloid polypeptide oligomers form intracellularly in the secretory pathway. Am J Pathol. 2010 February; 176(2):861-9. Epub 2009 Dec. 30.
11. Despa F (2009) Dilation of the endoplasmic reticulum in beta cells due to molecular overcrowding? Kinetic simulations of extension limits and consequences on proinsulin synthesis. Biophys. Chemyst. 140:115-121
12. Despa F. (2010) Endoplasmic reticulum overcrowding as a mechanism of beta-cell dysfunction in diabetes. Biophys. J. 98 1641-1648.
13. Huang C J, Gurlo T, Haataja L, Costes S, Daval M, Ryazantsev S, Wu X, Butler A E, Butler P C. Calcium-activated calpain-2 is a mediator of beta cell dysfunction and apoptosis in type 2 diabetes. J Biol Chem. 2010 Jan. 1; 285(1):339-48. Epub 2009 Oct. 27.
14. Lin C Y, Gurlo T, Kayed R, Butler A E, Haataja L, Glabe C G, Butler P C. (2007) Toxic Human IAPP Oligomers are Intracellular, and Vaccination to Induce Anti-toxic Oligomer Antibodies Does not Prevent Human IAPP Induced Beta-Cell Apoptosis in human IAPP transgenic mice. Diabetes 56(5):1324-32.
15. Mattson M P, Goodman Y (1995) Different amyloidogenic peptides share a similar mechanism of neurotoxicity involving reactive oxygen species and calcium. Brain Res 676:219-224;
16. Kawahara M, Kuroda Y, Arispe N, Rojas E (2000) Alzheimer's beta-amyloid, human islet amylin, and prion protein fragment evoke intracellular free calcium elevations by a common mechanism in a hypothalamic GnRH neuronal cell line. J Biol Chem 275:14077-14083
17. Abramov A Y, Canevari L, Duchen M R (2003) Changes in intracellular calcium and glutathione in astrocytes as the primary mechanism of amyloid neurotoxicity. J Neurosci 23:5088-5095;
18. Demuro A, Mina E, Kayed R, Milton S C, Parker I, Glabe C G (2005) Calcium dysregulation and membrane disruption as a ubiquitous neurotoxic mechanism of soluble amyloid oligomers. J Biol Chem 280:17294-17300
19. Bers D M. (2008) Calcium cycling and signaling in cardiac myocytes. Annu Rev Physiol. 70:23-49.
20. Bell D S, Diabetic cardiomyopathy. Diabetes Care. 2003; 26:2949-2951.
21. An, D. and Rodrigues, B. (2006). Role of changes in cardiac metabolism in development of diabetic cardiomyopathy. Am. J. Physiol. Heart Circ. Physiol. 291:H1489-H1506.
22. Poornima I G, Parikh P, Shannon R P (2006) Diabetic cardiomyopathy: the search for a unifying hypothesis. Circ Res 98:596-605.
23. Ashrafian H, Frenneaux M P, and Opie L H (2007) Metabolic Mechanisms in Heart Failure. Circulation 116: 434-448.
24. Boudina S, Abel E D, (2007) Diabetic cardiomyopathy revisited, Circulation. 115:3213-3223
25. Guha A, Harmancey R, Taegtmeyer H. (2008) Nonischemic heart failure in diabetes mellitus. Curr Opin Cardiol. 23:241-8.
26. Asghar, O., Al-Sunni, A., Khavandi, K., Khavandi, A., Withers, S., Greenstein, A., Heagerty, A. M. and Malik, R. A. (2009). Diabetic cardiomyopathy. Clin. Sci. 116:741-760.
27. Rutter M K, H. Parise and E. J. Benjamin, Levy D, Larson M G, Meigs J B, Nesto R W, Wilson P W, Vasan R S. (2003) Impact of glucose intolerance and insulin resistance on cardiac structure and function: sex-related differences in the Framingham Heart Study, Circulation 107:448-454
28. Ingelsson E, J. Sundstrom and J. Amlov, Zethelius B, Lind L. (2005) Insulin resistance and risk of congestive heart failure, J Am Med Assn 294:334-341.
29. Kamalesh M (2007) Heart Failure in Diabetes and Related Conditions, J Cardiac Failure 13:861-873.
30. Kostis J B, Sanders M (2005) The association of heart failure with insulin resistance and the development of type 2 diabetes. Am J Hypertens 18:731-7.
31. Reaven, G M (2005) Insulin resistance, the insulin resistance syndrome, and cardiovascular disease, Panminerva Med. 47:201-210.
32. Masoudi F A, Inzucchi S E (2007) Diabetes mellitus and heart failure: epidemiology, mechanisms, and pharmacotherapy. Am J Cardiol 99:113B-132B.
33. Cubbon R, Kahn M, Kearney M T (2008) Secondary prevention of cardiovascular disease in type 2 diabetes and prediabetes: a cardiologist's perspective. Int J Clin Pract 62:287-299.
34. Anselmino M, Gohlke H, Mellbin L and Ryden L (2008) Cardiovascular prevention in patients with diabetes and prediabetes. Herz, 33:170-177.
35. Swan J W, Anker S D, Walton C, Godsland I F, Clark A L, Leyva F, Stevenson J C, Coats A J (1997) Insulin resistance in chronic heart failure: relation to severity and etiology of heart failure, J Am Coll Cardiol 30:527-532
36. Ashrith G, Algahim M F, Taegtmeyer H. Insulin resistance: marker or mediator? Am J Med. 2009 March; 122(3): e13-e15.
37. Utriainen T, Takala T, Luotolahti M, Ronnemaa T, Laine H, Ruotsalainen U, Haaparanta M, Nuutila P, Yki-Jarvinen H. (1998) Insulin resistance characterizes glucose uptake in skeletal muscle but not in the heart in NIDDM. Diabetologia. 41:555-559
38. Jagasia D, Whiting J M, Concato J, Pfau S, McNulty P H (2001) Effect of non-insulin-dependent diabetes mellitus on myocardial insulin responsiveness in patients with ischemic heart disease. Circulation. 103:1734-1739
39. Kirkitadze, M. D.; Bitan, G.; Teplow, D. B. (2002) Paradigm shifts in Alzheimer's disease and other neurodegenerative disorders: the emerging role of oligomeric assemblies. J Neurosci Res. 69:567-577
40. Pereira L, Matthes J, Schuster I, Valdivia H H, Herzig S, Richard S, Gomez A M (2006) Mechanisms of [Ca2+]i transient decrease in cardiomyopathy of db/db type 2 diabetic mice. Diabetes 55:608-15
41. Belke, D. D., Swanson, E. A. and Dillmann, W. H. (2004) Decreased sarcoplasmic reticulum activity and contractility in diabetic db/db mouse heart. Diabetes 53,3201-3208
42. Yaras N, Ugur M, Ozdemir S, Gurdal H, Purali N, Lacampagne A, Vassort G, Turan B. Effects of diabetes on ryanodine receptor Ca release channel (RyR2) and Ca2+ homeostasis in rat heart. Diabetes. 2005 November; 54(11):3082-8.
43. Netticadan T, Temsah R M, Kent A, Elimban V, Dhalla N S. Depressed levels of Ca2+-cycling proteins may underlie sarcoplasmic reticulum dysfunction in the diabetic heart. Diabetes. 2001 September; 50(9):2133-8.
44. Bugger H, Abel E D (2008) Molecular mechanisms for myocardial mitochondrial dysfunction in the metabolic syndrome, Clin Sci (London) 114:195-210.
45. Maack C, O'Rourke B. Excitation-contraction coupling and mitochondrial energetics. Basic Res Cardiol. 2007 September; 102(5):369-92.
46. Giardino, I., Edelstein, D. and Brownlee, M. (1996) BCL-2 expression or antioxidants prevent hyperglycemia-induced formation of intracellular advanced glycation endproducts in bovine endothelial cells. J. Clin. Invest. 97, 1422-1428;
47. Nishikawa, T., Edelstein, D., Du, X. L, Yamagishi S, Matsumura T, Kaneda Y, Yorek M A, Beebe D, Oates P J, Hammes H P, Giardino I, Brownlee M. (2000) Normalizing mitochondrial superoxide production blocks three pathways of hyperglycaemic damage. Nature 404,787-790.
48. Hayden M R, Tyagi S C (2002) Islet redox stress: the manifold toxicities of insulin resistance, metabolic syndrome and amylin derived islet amyloid in type 2 diabetes mellitus. JOP 3:86-108.
49. Wendt T, Tanji N, Guo J, Hudson B I, Bierhaus A, Ramasamy R, Arnold B, Nawroth P P, Yan S F, D'Agati V, Schmidt A M (2003) Glucose, glycation, and RAGE: implications for amplification of cellular dysfunction in diabetes nephropathy. J Am Soc Nephrol 14: 1383-1395.
50. Simpson S H, Majumdar S R, Tsuyuki R T, Eurich D T, Johnson J A (2006) Dose-response relation between sulfonylurea drugs and mortality in type 2 diabetes mellitus: a population-based cohort study CMAJ. 174:169-74.
51. McAlister F A, Eurich D T, Majumdar S R, Johnson J A. (2008) The risk of heart failure in patients with type 2 diabetes treated with oral agent monotherapy Eur J Heart Fail. 10:703-8.
52. Westermark P, Benson M D, Buxbaum J N, Cohen A S, Frangione B, Ikeda S, Masters C L, Merlini G, Saraiva M J, Sipe J D. (2002) Amyloid fibril protein nomenclature. Amyloid 9:197-200.

INFORMAL SEQUENCE LISTING

SEQ ID NO: 1
Human IAPP amino acid sequence
KCNTATCATQRLANFLVHSSNNFGAILSSTNVGSNTY
SEQ ID NO: 2
Rat IAPP amino acid sequence
KCNTATCATQRLANFLVRSSNNLGPVLPPTNVGSNTY

1. A method for predicting a propensity for failure of an organ selected from heart and kidney in an individual who has type 2 diabetes or is pre-diabetic, the method comprising
determining the amount of islet amyloid polypeptide (IAPP) oligomer in a sample from the individual; and
predicting the propensity for heart failure in the individual based on the determined amount of IAPP oligomer, wherein an elevated amount of IAPP oligomer compared to normal levels indicates an increased propensity for heart failure.

2. The method of claim 1, wherein the sample is a blood sample extracted from said individual.

3. (canceled)

4. (canceled)

5. The method of claim 1, wherein the determining step comprises
contacting an antibody reagent that specifically binds IAPP oligomers to the sample; and
detecting the amount of IAPP oligomers bound by the reagent, said detecting comprising:
contacting a detectably labeled detection antibody that binds IAPP oligomers to the IAPP oligomers bound to the antibody reagent; and
quantifying the binding of the detection antibody to the bound IAPP oligomers.

6. (canceled)

7. (canceled)

8. (canceled)

9. (canceled)

10. The method of claim 1, wherein it is determined that the individual has a propensity for heart failure or kidney failure, the method further comprising designing a treatment plan to reduce the propensity for heart failure or kidney failure, respectively in the individual.

11. A kit for predicting a propensity for failure of an organ selected from heart and kidney in an individual who has type 2 diabetes or is pre-diabetic, the kit comprising a solid support operably linked to a reagent that specifically binds IAPP oligomers.

12. The kit of claim 11, wherein the reagent is an antibody.

13. The kit of claim 11, further comprising a detectably labeled detection antibody that binds to IAPP oligomers when the oligomers are bound to the reagent.

14. (canceled)

15. The kit of claim 11, wherein the solid support comprises a sensor operably linked to one or more nanoparticles, wherein the one or more nanoparticles are conjugated to an antibody that specifically binds IAPP oligomers.

16. A method for screening for agents that prevent or reduce the propensity for failure of on organ selected from heart and kidney in an individual who has type 2 diabetes or is pre-diabetic, the method comprising screening a plurality of agents for the ability:
to enhance excretion of IAPP oligomers from the body and/or
to block or interfere with the formation and/or function of IAPP oligomers.

17. The method of claim 16, further comprising identifying at least one agent from the plurality that enhances excretion of IAPP oligomers from the body and/or blocks or interferes with the formation of IAPP oligomers; and
administering the identified agent to an animal and measuring the ability of the agent to reduce the rate of failure of an organ selected from heart and kidney.

18. The method of claim 17, wherein the animal is an animal model for diabetes or prediabetes.

19. (canceled)

20. A method of treating or preventing failure of an organ selected from heart and kidney in an individual who has type 2 diabetes or is pre-diabetic, the method comprising
administering an effective amount of a compound that has the ability to
i) enhance excretion of IAPP oligomers from the body,
ii) block or interfere with the formation of IAPP oligomers, or
iii) block or interfere with the function of IAPP oligomers.

21. The method of claim 20, wherein the compound is a surfactant.

22. The method of claim 21, wherein the surfactant is a polymer-based membrane sealant.

23. The method of claim 22, wherein the polymer-based membrane sealant blocks or interferes with the function of IAPP oligomers by restoring membranes damaged by IAPP oligomers.

24. A method for predicting a propensity for failure of an organ selected from heart and kidney in an individual who is pre-diabetic, the method comprising
determining the amount of islet amyloid polypeptide (IAPP) oligomer in a sample from the individual; and
predicting the propensity for heart or kidney failure, respectively, in the individual based on the molecular weight bands corresponding to the amount of IAPP oligomer, wherein an elevated amount of larger molecular weight IAPP oligomers compared to smaller molecular weight IAPP oligomers indicates an increased propensity for heart or kidney failure.

25. The method of claim 24, wherein an elevated amount of smaller molecular weight IAPP oligomers indicates a likelihood of accumulating larger molecular weight IAPP oligomers.

26. The method of claim 24, wherein the smaller molecular weight IAPP oligomers are about 12 or 16 kDa.

27. The method of claim 24, wherein the larger molecular weight IAPP oligomers are about 32 or 64 kDa.

28.-50. (canceled)


SEQUENCE LISTING (2 sequences)

SEQ ID NO: 1 - protein, 36 aa, Homo sapiens
KCNTATCATQLANFLVHSSNNFGAILSSTNVGSNTY

SEQ ID NO: 2 - protein, 36 aa, Mus musculus
KCNTATCATQLANFLVRSSNNLGPVLPPTNVGSNTY